(19) United States
(12) Patent Application Publication
(10) Pub. No.: US 2006/0246513 A1
(43) Pub. Date: Nov. 2, 2006

(54) Method and device to detect the presence of analytes in a sample

(76) Inventor: Robert C. Bohannon, Chapel Hill, NC (US)

Correspondence Address: NEEDLE & ROSENBERG, P.C., SUITE 1000, 999 PEACHTREE STREET, ATLANTA, GA, 30309-3915, US

(21) Appl. No.: 11/415,520

(22) Filed: May 2, 2006

Related U.S. Application Data
(60) Provisional application No. 60/676,703, filed on May 2, 2005.

(51) Int. Cl. G01N 33/53; C12M 1/34

(52) U.S. Cl. 435/7.1; 435/287.2

(57) ABSTRACT

Disclosed are methods and apparatus useful for determining the presence or absence of one or more analytes in a liquid sample, such as a biological or environmental sample. In some embodiments, the method can use an indirect competitive immunochromatagraphic test strip.

### CROSS-REFERENCE TO RELATED APPLICATIONS

This application claims benefit of U.S. Provisional Application No. 60/676,703, filed May 2, 2005. application Ser. No. 60/676,703, filed May 2, 2005, is hereby incorporated herein by reference in its entirety.

### BACKGROUND OF THE INVENTION

This invention relates to analyte detection. More particularly, the invention relates to methods and apparatus for the detection of analytes in a liquid sample such as a biological or environmental sample.

Many types of analyte-receptor assays have been used to detect the presence of various substances, often generally called analytes, in body fluids. These assays involve antigen antibody reactions, synthetic conjugates comprising radioactive, enzymatic, fluorescent, or visually observable metal sol tags, and specially designed reactor chambers. In all these assays, there is a receptor, e.g., an antibody, which is specific for the selected analyte or antigen, and a means for detecting the presence, and often the amount, of the analyte-receptor reaction product. Most current tests are designed to make a quantitative determination, but in many circumstances all that are required is a positive/negative indication. Examples of such qualitative assays include blood typing and most types of urinalysis. For these tests, visually observable indicia such as the presence of agglutination or a color change are preferred.

Even the positive/negative assays must be very sensitive because of the often small concentration of the analyte in the test fluid. False positives can also be troublesome, particularly with agglutination and other rapid detection methods such as dipstick and color change tests. Because of these problems, sandwich assays and other sensitive detection methods which use metal sols or other types of colored particles have been developed. These techniques have not solved all of the problems encountered in these rapid detection methods.

There are two basic formats for lateral flow detection tests. In the traditional lateral flow format the substance being detected is collected in a liquid then applied to an absorbent sample pad where visually colored particles bind specifically to the target of interest thereby labeling it with a color so that it can be visually read without the need of an instrument. The labeled target flows by capillary action along a porous membrane and is subsequently trapped by another material affixed to the membrane at a designated location, which also binds the labeled target, until sufficient colored particles accumulate at the defined location thereby giving a visual signal readable by the user. The development of a colored band indicates the presence of the target in the sample being tested.

The second format typically used to make lateral flow tests is similar to the capture method described above, but uses a competitive method whereby the antibody labeled particle on the sample pad competes for either the target in the sample or a similar target bound at a defined position as the liquid sample and colored particles flow across the porous membrane. In this format, if the target is present in the sample, it pre-binds the labeled particle thereby inhibiting the labeled particles to bind at the defined location. In this format, the loss of a visual color at the defined location means there is a sufficient amount of the target in the sample to inhibit the color change. If the sample contains no target, a visual line forms at the defined location. This is the format typically used by most companies to detect drugs of abuse, but which is counter-intuitive to most untrained users.

It is an object of this invention to provide a method and apparatus for detecting analytes. Another object is to provide a method and apparatus for detecting analytes in a competitive format where the presence of the analyte gives a positive signal. Another object is to provide a lateral flow method and apparatus for detecting analytes that can provide quantitative results. Another object is to provide a method and apparatus for detecting analytes that includes a control test. Another object is to provide a test cell for detection of analytes. Another object is to provide a test cell for detection of analytes in a competitive format where the presence of the analyte gives a positive signal. Another object is to provide a test cell for detection of large molecules. Another object is to provide an assay system which involves a minimal number of procedural steps, and yields reliable results.

These and other objects and features of the invention will be apparent from the following description, drawings, and claims.

### BRIEF SUMMARY OF THE INVENTION

Disclosed are methods and apparatuses, such as test cells, for the detection of one or more analytes. The disclosed methods and apparatuses are particularly useful for detecting analytes in a liquid sample such as a biological or environmental sample. Virtually any analyte in any sample can be detected using the disclosed method and apparatus. The disclosed method can be performed in numerous formats. For example, multiple analytes can be detected in the same, different, or combination of apparatuses; multiple analytes can be detected in the same, different or combination of assays; and multiple analytes can be detected simultaneously, sequentially, or in any temporal order. The disclosed method makes use of both a competitive assay element and a capture assay element. This combination has the benefit of producing a visible signal in the presence of an analyte while using the format of a competitive assay.

The disclosed method generally involves, in the presence of one or more samples suspected of containing one or more analytes, (1) binding of one or more labeled agents (LA) to one or more moderation capture agents (MCA) in the absence of one or more of the analytes and no binding or reduced binding of the labeled agents to the moderation capture agents in the presence of one or more of the analytes, and (2) binding of labeled agents that are not bound to moderation capture agents to one or more test capture agents (TCA). The labeled agent can be detected. Such detection indicates the presence of one or more of the analytes in one or more of the samples because labeled agents become available to bind test capture agents when the labeled agents fail to bind moderation capture agents in the presence of the analytes. The first aspect of the method is the competitive assay element of the disclosed method and the second aspect is the capture assay element of the disclosed method.

In useful forms of the method, the moderation capture agents and test capture agents can be immobilized at different physical locations and they can be exposed to the labeled agents and samples sequentially. For example, a liquid flow of combined sample and labeled agent can flow through one or more moderation zones (where one or more moderation capture agents can be immobilized) and then through one or more test zones (where one or more test capture agents can be immobilized). In a liquid flow format, the mobile components (for example, analytes and labeled agents) can be transported by any suitable means such as by capillary flow, electrophoretic force, gravity (natural or artificial), and chromatographic flow. In some forms of the method, the method can be performed using one or more test cells, where a liquid sample can flow along the cell and encounter the different components and immobilization areas in sequence. An example of a useful test cell is a lateral flow test strip.

The disclosed method generally can be performed, and the disclosed apparatus generally can be configured, in two main formats. In one format, which can be referred to as the MCA/analyte competitive format (or MCA format), analyte and moderation capture agent (which can be similar to each other) compete with each other for binding to the labeled agent. Examples 1 and 3 illustrate this format. In the other format, which can be referred to as the LA/analyte competitive format (or LA format), analyte and labeled agents compete with each other for binding to the moderation capture agent. Examples 2 and 4 illustrate this format. In the LA format, an analyte (if present in a sample) competes with a labeled agent for binding to a moderation capture agent. Labeled agent binds to the moderation capture agent in the absence of analyte. In the presence of analyte, the analyte binds to the moderation capture agent, thus reducing or eliminating binding of the labeled agent to the moderation capture agent. Labeled agent that does not bind to moderation capture agent can then go on to bind test capture agent. Thus, detection of the labeled agent bound to test capture agent (that is, in the test zone) indicates the presence of analyte. An example of an embodiment of the LA format is shown in FIG. 3, where the analyte is a drug of abuse competing with a labeled agent containing the same drug of abuse, a mimic of the drug, or an analogue of the drug to bind to the moderation capture agent, which is a monoclonal antibody to the drug of abuse being assayed for, or a mimic or an analogue compound of the drug.

In the MCA format, an analyte (if present in a sample) competes with a moderation capture agent for binding to a labeled agent. Labeled agent binds to the moderation capture agent in the absence of analyte. In the presence of analyte, the analyte binds to the labeled agent, thus reducing or eliminating binding of the labeled agent to the moderation capture agent (the labeled agent cannot bind, or can only bind with less affinity, to the moderation capture agent when the labeled agent is bound to the analyte). Labeled agent that does not bind to moderation capture agent can then go on to bind test capture agent. Thus, detection of the labeled agent bound to test capture agent (that is, in the test zone) indicates the presence of analyte. An example of an embodiment of the MCA format is shown in FIG. 4. Competition for binding can be accomplished by including molecules, elements or moieties in the moderation capture agent that are similar or identical to molecules, elements or moieties of the analyte that belongs to the same test set as the moderation capture agent (that is, the analyte with which the moderation capture agent competes for binding to the labeled agent). A test set is a complementary set of reagents used to detect a specific analyte or group of analytes. In the MCA format, useful moderation capture agents can be the analyte that belongs to the same test set as the moderation capture agent, an analyte mimic or mimetic of the analyte that belongs to the same test set as the moderation capture agent, or a derivative of the analyte that belongs to the same test set as the moderation capture agent. In some instances, the detection of the analyte may be done using indirect methods, such as adding a monoclonal anti-drug of abuse antibody to the sample or upstream of the label zone (in FIG. 4, for example) and employing a labeled agent that binds the monoclonal to the drug, such as protein A labeled with colloidal gold particles, thereby allowing the label to participate in the reaction of the antibody bind to the drug or to the moderation capture agent, which contains the drug of interest or an analogue or mimic thereof. Hence, indirect detection of analyte can be done with the label or with the moderation zone in a similar fashion and is contemplated herein.

The method and apparatus can also make use of one or more sample zones, where the sample can be brought into contact with the flow path or substrate. The sample zone can be upstream of the moderation zone relative to the flow of the sample. The method and apparatus can also make use of one or more label zones. The labeled agents can be detachably localized or detachably immobilized to the substrate in at least one of the label zones. Contact by the sample with the labeled agents can cause the labeled agents to detach and flow with the sample. The label zone can be downstream of the sample zone, relative to the flow of the sample. The label zone can be upstream of the moderation zone relative to the flow of the sample. The method and apparatus can also make use of one or more reservoirs. The reservoir can be downstream of the test zone relative to the flow of the sample. The reservoir can receive the sample that flows through the substrate. The various zones can be visible, observable, or detectable or not visible, observable, or detectable outside of the test cell. For example, the moderation zone can be visible, observable, or detectable or not visible, observable, or detectable outside of the test cell.

Where multiple analytes, multiple labeled agents, multiple moderation capture agents, multiple test capture agents, or any combination are used in the same embodiment of the disclosed method or apparatus, these two formats can be combined in that embodiment (for multiple analytes, multiple labeled agents, multiple moderation capture agents, and/or multiple test capture agents used in one or both of the formats). Where multiple moderation zones and/or multiple test zones are used, the various zones can have a variety of spatial relationships. For example, multiple moderation zones can be placed sequentially, stacked on top of each other, and/or located adjacently along the flow path, with each moderation zones placed upstream of the test zone(s) that belongs to the same test set as the moderation zone. As another example, moderation zones can be interspersed, alternated, or both interspersed and alternated with test zones. In such cases, the test zone(s) that belong to the same test set as particular moderation zone(s) generally should be placed downstream of the moderation zone(s) that belongs to the same test set. A test zone belongs to the same test set as a moderation zone when the moderation zone includes at least one moderation capture agent that belongs to the same test set as at least one test capture agent in the test zone. A moderation capture agent belongs to the same test set as a test capture agent when the moderation capture agent and test capture agent can both bind the same labeled agent, directly or indirectly. The various zones can have a variety of geometric configurations.

The disclosed method can use labeled agents comprising a binding agent and a label, wherein the label is bound, coupled to or associated with the binding agent. The binding agent of a labeled agent mediates binding of the labeled agent to the moderation capture agent that belongs to the same test set as the labeled agent. The binding agent can comprise, for example, a binding region for the moderation capture agent that belongs to the same test set as the labeled agent. This binding region for the moderation capture agent can be referred to as the moderation binding region.

In some embodiments of the disclosed method, a liquid sample (which can also be referred to as a test liquid) can be mixed with the labeled agent outside the test cell or prior to bringing the sample into contact with the test cell. In other embodiments, the labeled agent can be disposed in the substrate of the test cell between the inlet and the moderation zone. For example, the labeled agent can be in freeze-dried or other preserved form and/or combined with stabilizing agents on the substrate between the inlet and the moderation zone. The labeled agent can be disposed in the substrate such that the liquid sample can resolubilize the labeled agent as it passes along the flow path.

In some embodiments, the amount or level of signal detected in the test zone can be compared with the amount or level of signal detected in the moderation zone. Depending on the amount of analyte present (and on the relative amount of analyte, labeled agent and moderation capture agent), all, substantially all, some, substantially none, or none of the labeled agent may bind to the moderation capture agent. Thus, the amount of labeled agent that accumulates in the moderation zone depends on the amount of analyte in the sample. Depending on the relative amount of labeled agent and test capture agent, some, substantially all, or all of the labeled agent that does not bind the moderation capture agent will bind the test capture agent. The ratio of the amount of labeled agent accumulated at the moderation zone as compared to the amount accumulated at the test zone can be proportional to the presence of the analyte. Hence, the more analyte present in the sample, the more label that accumulates within the test zone and less label that accumulates in the moderation zone. The ratio of label detected in the moderation zone to label detected in the test zone can provide information about the amount of analyte present in the sample and can serve as an internal control that the expected interactions are taking place in the method.

Label detected in the test zone (and/or moderation zone) can be compared with label of one or more standards or internal controls to determine whether the label detected is a true indication of the presence or absence of the analyte, or an artifact caused by interfering conditions. For example, high or low pH, the presence of detergent, the presence of cross-linking agents in samples (such as glutaraldehyde) can affect or prevent or alter the interactions and bindings for which the method is designed.

One control format generally involves use of one or more control labeled agents (CLA) that can bind to one or more control capture agents (CCA). Binding of the control labeled agent to the control capture agent can serve as an indication that binding can occur in the samples being used. The control labeled agent can comprise a binding agent and a label. The label of the control labeled agent that is bound to control capture agent can be detected. Such detection indicates that binding can occur in the samples being used and can provide assurance that non-detection of analyte in the method is accurate and not due to some interfering condition in the samples.

The control capture agents can be immobilized in one or more control zones. In forms of the method, performed using one or more test cells, the sample zones generally can be upstream of the control zones. For ease of detection, it is useful to dispose control zones in different locations than test zones. However, control zones and test zones also can be partially or fully overlapping. In cases where a control zone fully overlaps with a test zone, it is useful to use labels on the labeled agents and control labeled agents that can be separately detected or whose detection can be distinguished. Where multiple control zones are used, the zones can have a variety of spatial relationships to each other and to other zones (such as moderation zones and test zones). Control zones can have a variety of geometric configurations.

The disclosed method can use a control labeled agent comprising a binding agent and a label, wherein the label is bound, coupled to or associated with the binding agent. The binding agent of a control labeled agent mediates binding of the control labeled agent to the control capture agent that belongs to the same test set as the control labeled agent. The binding agent can comprise, for example, a binding region for the control capture agent that belongs to the same test set as the control labeled agent. This binding region for the control capture agent can be referred to as the control moderation binding region.

The label in labeled agents and control labeled agents can be detected by any appropriate means, mode or manner. Generally, the mode or manner of detection can be based on the label used and the type of signal that the label generates. For example, some labels generate or produce radiation or a field that can be detected. Such radiation or field can be an added property of the label, can be a characteristic of the structure of the label, or both. Such radiation or field can also be generated by the combination of the label and some other component, or by a component that is associated with the label, generated by the label, or whose generation is mediated by the label. These latter modes can be referred to as indirect signal generation. Labels can also be detected by physical analysis of all or part of the label. For example, labels or portions of labels (or even entire labeled agents) can be analyzed by mass spectrometry. For this type of detection, useful labels can include mass tags, include multiple mass tags that can be distinguished form each other by mass spectrometry. Many types of signal, many methods for generating signals, and many methods of detecting signals are known and can be use with and adapted for the disclosed methods and apparatuses. Some signals can be detected visually and these and most other signals can be detected through the use of appropriate instruments. For example, the label in a labeled agent or control labeled agent can be detected by visual observation of label development at the test zone or control zone in the test cell (for labels that produce a visual signal) and/or by the use of an instrument to detect, for example, any radiation or field that may be generated by the label.

In some embodiments of the disclosed method, a liquid sample can be mixed with the control labeled agent outside the test cell or prior to bringing the sample into contact with the test cell. In other embodiments, the control labeled agent can be disposed in the substrate of the test cell, preferably between the inlet and the moderation zone. One or more control capture agents can be immobilized in one or more control zones of one or more test cells.

Other control formats can include (along with control labeled agents and control capture agents) one or more control analytes (CA) and/or one or more control moderation capture agents (CMCA). Generally, in these formats, control analytes, control labeled agents, control moderation capture agents and control capture agents can be used in the same ways and with the same relationships as analytes, labeled agents, moderation capture agents, and test capture agents. In some embodiments, the amount or level of signal detected in the control zone can be compared with the amount or level of signal detected in the control moderation zone.

Useful apparatuses for use with the disclosed method include test cells. Test cells are engineered to comprise a flow path through which liquid samples can flow, which can be, for example, through a porous material and/or via channels. Generally the flow path of a test cell can comprise at least one moderation zone and at least one test zone. The test cell and flow path can be constructed of any suitable material. Generally the flow path can comprise a void space and/or a material through which liquid or fluid can flow or is directed to flow. The test cell can consist of the flow path or can further comprise other elements and structures. For example, useful test cells comprise a body, which can be unitary or multipart, in which the flow path is disposed. The body can comprise one or more inlets where one or more fluids can be introduced to the test cell. Flow path material can also extend outside the body of the flow cell and such flow path material can be used to introduce fluid to the test cell.

Useful flow paths can comprise a permeable material through which liquid or fluid can flow and/or can comprise impermeable material, such as a channel, that directs the flow of the liquid. The flow path can be unitary or multipart. For example, flows paths can comprise a single substantially homogenous material, a single heterogeneous material, multiple different elements of the same materials, multiple different elements of different materials, or any combination. For example, a flow path can comprise a single element. Different components for use in the disclosed method can be disposed in different regions of this single element. As another example, a flow path can comprise a base material in which or on which one or more pads are located. In such embodiments, it is useful to provide different components for use in the disclosed method on different pads. For example, one or more pads can comprise or embody one or more moderation zones, one or more test zones, one or more control zones, one or more control moderation zones, one or more reservoir zones, and/or one or more sample zones.

Some forms of test cell can have reservoir zones and/or windows. The reservoir zone can act to receive liquid transported along the flow path defined by the permeable material and/or liquid channeling material and extending from the inlet and through the test volume. Windows in the body of test cells can be solid, open, transparent, opaque or in other condition. Detection need not require a window. For detection purposes, all that is required is that measurement or detection of label be possible. Thus, for example, a visual observation can make use of a transparent or translucent window. A radiation or field detection by an instrument may not require any window if the radiation or field can penetrate the body of the test cell. Alternatively, the window can be made of a material transparent or translucent to the radiation or field to be detected. A window can also be an uncovered opening onto the flow path. A window can also be open or exposed or can be openably covered with some other material. Test cells can have other features and configurations.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic representation of a test cell.

FIG. 2 shows a schematic representation of a test cell.

FIG. 3 shows a schematic representation of an ICS antigen competitive strip (LA format).

FIG. 4 shows a schematic representation of an ICS small molecule competitive strip (MCA format).

FIG. 5 shows a schematic representation of the LA format of disclosed method.

FIG. 6 shows a schematic representation of the MCA format of disclosed method.

FIG. 7 shows a schematic representation of the LA format of disclosed method in the absence of analyte.

FIG. 8 shows a schematic representation of the MCA format of disclosed method in the absence of analyte.

FIG. 9 shows a schematic representation of the LA format of disclosed method with a control.

FIG. 10 shows a schematic representation of the LA format of disclosed method with a control.

### DETAILED DESCRIPTION OF THE INVENTION

The disclosed method and compositions may be understood more readily by reference to the following detailed description of particular embodiments and the Example included therein and to the Figures and their previous and following description.

Disclosed are methods and apparatuses, such as test cells, for the detection of one or more analytes. The disclosed methods and apparatuses are particularly useful for detecting analytes in, for example, a liquid sample such as a biological or environmental sample. Virtually any analyte in any sample can be detected using the disclosed method and apparatus. For example, proteins, peptides, antibodies, small molecules, metabolites, hormones, breakdown products, antigens, epitopes, nucleic acids, lipids, carbohydrates, sugars, cells, spores, viruses, bacteria, inorganic compounds, organic compounds, drugs, poisons, contaminants, and the like are analytes that can be detected with the disclosed methods and apparatuses. The disclosed method can be performed in numerous formats. For example, multiple analytes can be detected in the same, different, or combination of apparatuses; multiple analytes can be detected in the same, different or combination of assays; and multiple analytes can be detected simultaneously, sequentially, or in any temporal order. The disclosed method makes use of both a competitive assay element and a capture assay element. This combination has the benefit of producing a visible signal in the presence of an analyte while using the format of a competitive assay.

The disclosed method generally involves, in the presence of one or more samples suspected of containing one or more analytes, (1) binding of one or more labeled agents (LA) to one or more moderation capture agents (MCA) in the absence of one or more of the analytes and no binding or reduced binding of the labeled agents to the moderation capture agents in the presence of one or more of the analytes, and (2) binding of labeled agents that are not bound to moderation capture agents to one or more test capture agents (TCA). The labeled agent comprises a binding agent and a label. The label of the labeled agent that is bound to test capture agent can be detected. Such detection indicates the presence of one or more of the analytes in one or more of the samples because labeled agents become available to bind test capture agents when the labeled agents fail to bind moderation capture agents in the presence of the analytes. The first aspect of the method is the competitive assay element of the disclosed method and the second aspect is the capture assay element of the disclosed method.

In useful forms of the method, the moderation capture agents and test capture agents can be immobilized at different physical locations and they can be exposed to the combination of labeled agents and samples sequentially. For example, a liquid flow of combined sample and labeled agent can flow through one or more moderation zones (where one or more moderation capture agents can be immobilized) and then through one or more test zones (where one or more test capture agents can be immobilized). In a liquid flow format, the mobile components (for example, analytes and labeled agents) can be transported by any suitable means such as by capillary flow, electrophoretic force, gravity (natural or artificial), and chromatographic flow. The moderation capture agents can be immobilized in one or more moderation zones. The test capture agents can be immobilized in one or more test zones. In some forms of the method, the method can be performed using one or more test cells, where a liquid sample can flow along the cell and encounter the different components and immobilization areas in sequence. The samples can be brought into contact with test cells in an inlet, loading zone, or sample zone, which generally can be upstream of the moderation zones (which itself generally can be upstream of the test zones). An example of a useful test cell is a lateral flow test strip.

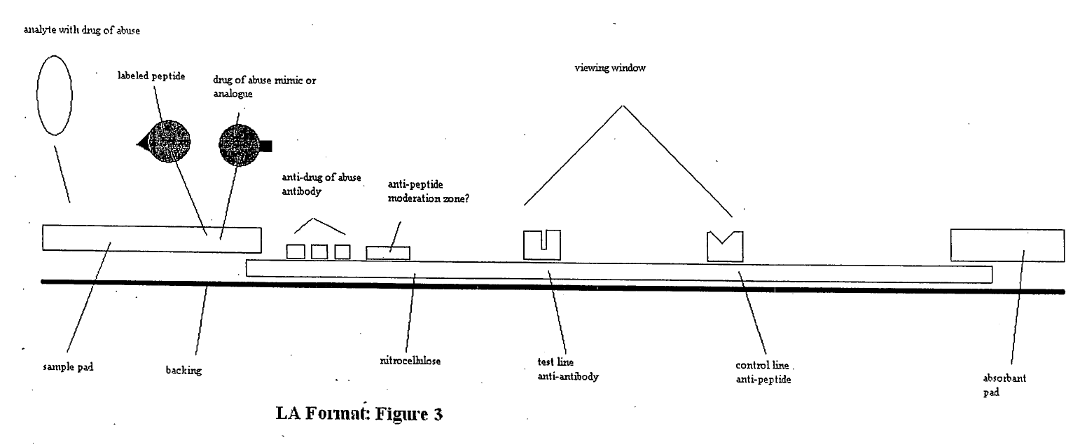

The disclosed method generally can be performed, and the disclosed apparatus generally can be configured, in two main formats. In one format, which can be referred to as the MCA/analyte competitive format (or MCA format), analyte and moderation capture agent (which can appear similar to each other) compete with each other for binding to the labeled agent (for example, antibody). Examples 1 and 3 illustrate this format. In the other format, which can be referred to as the LA/analyte competitive format (or LA format), analyte and labeled agents (which can appear similar to each other) compete with each other for binding to the moderation capture agent (for example, antibody). Examples 2 and 4 illustrate this format. In the LA format, an analyte (if present in a sample) competes with a labeled agent for binding to a moderation capture agent. Labeled agent binds to the moderation capture agent in the absence of analyte. In the presence of analyte, the analyte binds to the moderation capture agent, thus reducing or eliminating binding of the labeled agent to the moderation capture agent. Labeled agent that does not bind to moderation capture agent can then go on to bind test capture agent. Thus, detection of the labeled agent bound to test capture agent (that is, in the test zone) indicates the presence of analyte. An example of an embodiment of the LA format is shown in FIG. 3. Competition for binding can be accomplished by including molecules, elements or moieties in the labeled agents that are similar or identical to molecules, elements or moieties of the analyte that belongs to the same test set as the labeled agent. In the LA format, useful binding agents in the labeled agent can be the analyte that belongs to the same test set as the labeled agent, an analyte mimic or mimetic of the analyte that belongs to the same test set as the labeled analyte, or a derivative of the analyte that belongs to the same test set as the labeled agent.

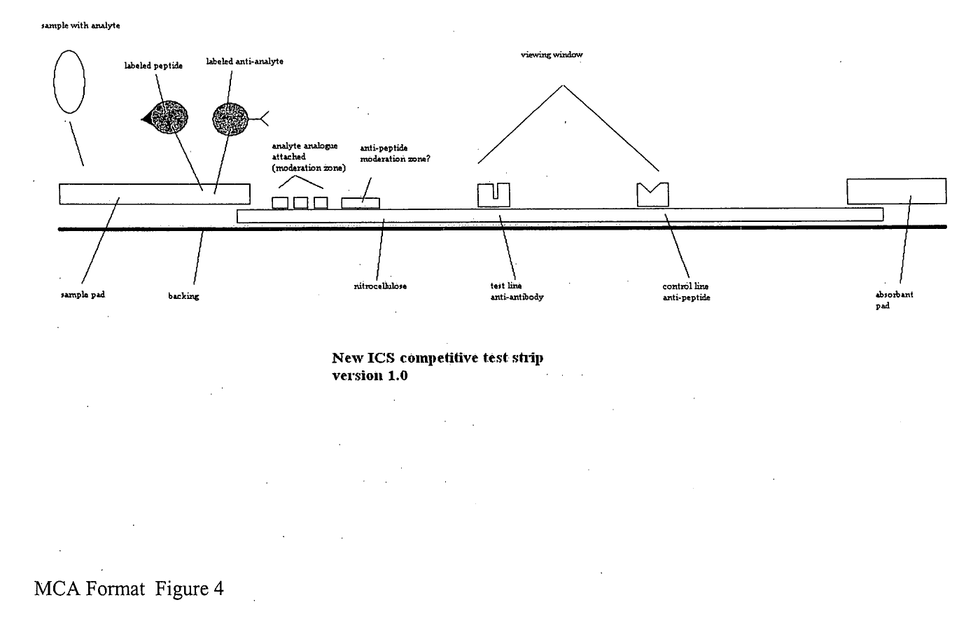

In the MCA format, an analyte (if present in a sample) competes with a moderation capture agent for binding to a labeled agent. Labeled agent binds to the moderation capture agent in the absence of analyte. In the presence of analyte, the analyte binds to the labeled agent, thus reducing or eliminating binding of the labeled agent to the moderation capture agent (the labeled agent cannot bind, or can only bind with less affinity, to the moderation capture agent when the labeled agent is bound to the analyte). Labeled agent that does not bind to moderation capture agent can then go on to bind test capture agent. Thus, detection of the labeled agent bound to test capture agent (that is, in the test zone) indicates the presence of analyte. An example of an embodiment of the MCA format is shown in FIG. 4. Competition for binding can be accomplished by including molecules, elements or moieties in the moderation capture agent that are similar or identical to molecules, elements or moieties of the analyte that belongs to the same test set as the moderation capture agent. In the MCA format, useful moderation capture agents can be the analyte that belongs to the same test set as the moderation capture agent, an analyte mimic or mimetic of the analyte that belongs to the same test set as the moderation capture agent, or a derivative of the analyte that belongs to the same test set as the moderation capture agent.

The operations in the disclosed method generally occur sequentially in the method, preferably via a directional flow of liquid. The result is that, in the absence of analyte, the labeled agent becomes immobilized at the location of the moderation capture agent (that is, the moderation zone). In the presence of analyte, the labeled agent becomes immobilized at the location of the test capture agent (that is, the test zone). The location of the label of the labeled agent thus indicates the presence or absence of the analyte. The presence of labeled agent in the moderation zone provides one form of negative control.

The method and apparatus can also make use of one or more sample zones, where the sample can be brought into contact with the flow path or substrate. The sample zone can be upstream of the moderation zone relative to the flow of the sample. The method and apparatus can also make use of one or more label zones. The labeled agents can be detachably localized or detachably immobilized to the substrate in at least one of the label zones. Contact by the sample with the labeled agents can cause the labeled agents to detach and flow with the sample. The label zone can be downstream of the sample zone, relative to the flow of the sample. The label zone can be upstream of the moderation zone relative to the flow of the sample. The method and apparatus can also make use of one or more reservoirs. The reservoir can be downstream of the test zone relative to the flow of the sample. The reservoir can receive the sample that flows through the substrate or fluidic channels. The various zones can be visible, observable, or detectable or not visible, observable, or detectable outside of the test cell. For example, the moderation zone can be visible, observable, or detectable or not visible, observable, or detectable outside of the test cell.

Where multiple analytes, multiple labeled agents, multiple moderation capture agents, multiple test capture agents, or any combination are used in the same embodiment of the disclosed method or apparatus, these two formats can be combined in that embodiment (for multiple analytes, multiple labeled agents, multiple moderation capture agents, and/or multiple test capture agents used in one or both of the formats). The format depends on the relationships between analytes, labeled agents and moderation capture agents that belong to the same test set. As used herein, a labeled agent belongs to the same test set as a moderation capture agent (and vice versa) when the moderation capture agent can bind the labeled agent in the absence of the analyte. As used herein, an analyte belongs to the same test set as a labeled agent and a moderation capture agent (and vice versa) when the analyte interferes with the binding of the labeled agent to the moderation capture agent by competing with the labeled agent for binding to the moderation capture agent or by competing with the moderation capture agent for binding to the labeled agent. Such grouping of test set components generally can be determined in the context of particular embodiments of the disclosed method and apparatus and need not be based on permanent or absolute relationships between the moderation capture agents, test capture agents, and labeled agents.

Where multiple moderation zones and/or multiple test zones are used, the various zones can have a variety of spatial relationships. For example, multiple moderation zones can be placed sequentially and adjacently along the flow path, with all moderation zones placed upstream of the test zone(s). As another example, moderation zones can be interspersed, alternated, or both interspersed and alternated with test zones. In such cases, the test zone(s) that belongs to the same test set as particular moderation zone(s) generally should be placed downstream of the moderation zone(s) that belongs to the same test set as the test zone(s). As used herein, a moderation zone belongs to the same test set as a test zone (and vice versa) when the moderation zone includes at least one moderation capture agent that belongs to the same test set as at least one test capture agent in the test zone. Thus, for example, moderation zones and test zones can be alternated with each moderation zone and test zone that belongs to the same test set adjacent to each other. As used herein, a moderation capture agent belong to the same test set as a test capture agent (and vice versa) when the moderation capture agent and test capture agent can both bind the same labeled agent, directly or indirectly. As used herein, a labeled agent belongs to the same test set as a test capture agent (and vice versa) when the test capture agent can bind the labeled agent, directly or indirectly. As used herein, an analyte belongs to the same test set as a test capture agent (and vice versa) when the analyte belongs to the same test set as a moderation test agent and/or a labeled agent that belong to the same test set as the test capture agent. Such groupings of test set components (analyte, moderation capture agent, labeled agent, test capture agent, and any other associated reagents or conditions that contribute to the specific detection of the analyte or group of analytes of interest) generally can be determined in the context of particular embodiments of the disclosed method and apparatus and need not be based on permanent or absolute relationships between the moderation capture agents, test capture agents, and labeled agents.

The various zones can have a variety of geometric configurations. For example, label, moderation, test, control, control label, and control moderation zones can be lines, bars, open or closed triangles, open or closed rectangles, open or closed squares, open or closed circles, open or closed ellipses, open or closed ovals, open or closed regular polygons, open or closed irregular polygons, open or closed amorphous shapes, spots, rings, cubes, spheres, pyramids, rectangular solids, or any other regular or irregular shape or solid. Label, moderation, test, control, control label, and control moderation zones need not have clear or distinct boundaries. Thus, for example, moderation capture agents can be immobilized in a regular or irregular pattern of spots with regular or irregular spaces in between. The area where the moderation capture agents are immobilized, optionally including or excluding the spaces in between, can be considered a moderation zone. The same is true of any other disclosed zones.

The disclosed method can use a labeled agent comprising a binding agent and a label, wherein the label is bound, coupled to or associated with the binding agent. The binding agent of a labeled agent mediates binding of the labeled agent to the moderation capture agent (and to the analyte in the MCA format). The binding agent can comprise, for example, a binding region for the moderation capture agent. This binding region for the moderation capture agent can be referred to as the moderation binding region. For use in an MCA format the binding region generally can be for the analyte that belongs to the same test set as the moderation capture agent. The labeled agent also comprises a binding region for the test capture agent. This binding region for the test capture agent (which can be referred to as the test binding region) can be any part or portion of the labeled agent and need not be (but can be or can overlap with) the moderation binding region. The test binding region need not be part of the binding agent of the labeled agent. The test binding region can be a component of the labeled agent present for that purpose. Such a component comprising the test binding region can be referred to as a test binding agent. A test binding agent can be, for example, a marker reagent, such as the blocking agent used in making the labeled agent, an oligonucleotide, or any other component that can be specifically bound. Alternatively, in the MCA format where an analyte-labeled agent conjugate forms, the labeled agent can bind to the test capture agent indirectly via the analyte. Where the test capture agent binds to the analyte in the analyte-labeled agent conjugate, a “sandwich” of the test capture agent, analyte, and the labeled agent can form. The test capture agent can also bind to a structure that is a combination part of the analyte and part of the labeled agent.

In some forms of the MCA format, analyte in a liquid sample is brought into contact with the labeled agent. As the labeled agent is exposed to the analyte, it binds the analyte, forming an analyte-labeled agent conjugate (which can be referred to as analyte-LA conjugate). As the liquid sample is transported into contact with the moderation zone, the moderation capture agent (which can, for example, mimic the analyte) within the moderation zone competes with the analyte for binding to the labeled agent. If no analyte is present, the labeled agent will bind to the moderation capture agent and aggregate at the moderation zone, thus resulting in an aggregation of label at the moderation zone. If the analyte is present, the amount of unconjugated labeled agent (that is, unconjugated with the analyte) that can bind at the moderation zone is reduced or null and the analyte-labeled agent conjugate continues to flow through the flow path toward the test zone. Once the analyte-labeled agent conjugate reaches the test zone, the analyte-labeled agent conjugate can react with the test capture agent of the test zone to form a labeled agent-test capture agent conjugate (which can be referred to as LA-TCA conjugate). The LA-TCA conjugate can be detected in any manner appropriate for the label used. The LA-TCA conjugate is progressively produced at the test zone as sample continuously passes by. As more and more analyte-labeled agent conjugate is immobilized, the label aggregates at the test zone. Detection of the label in the test zone indicates the presence of the analyte in the liquid sample.

In some forms of the LA format, analyte in a liquid sample is brought into contact with the labeled agent. As the liquid sample is transported into contact with the moderation zone, the labeled agent (which can, for example, mimic the analyte) competes with the analyte for binding to the moderation capture agent in the moderation zone. If no analyte is present, the labeled agent will bind to the moderation capture agent and aggregate at the moderation zone, thus resulting in an aggregation of label at the moderation zone. If the analyte is present, the amount of labeled agent that can bind at the moderation zone is reduced or null (because the analyte occupies the moderation capture agent) and the labeled agent continues to flow through the flow path toward the test zone. Once the labeled agent reaches the test zone, the labeled agent can react with the test capture agent of the test zone to form a labeled agent-test capture agent conjugate (LA-TCA conjugate). The LA-TCA conjugate can be detected in any manner appropriate for the label used. The LA-TCA conjugate is progressively produced at the test zone as sample continuously passes by. As more and more labeled agent is immobilized, the label aggregates at the test zone. Detection of the label in the test zone indicates the presence of the analyte in the liquid sample.

The label in the labeled agent can be detected by any appropriate means, mode or manner. Generally, the mode or manner of detection can be based on the label used and the type of signal that the label generates. For example, some labels generate or produce radiation or a field that can be detected. Such radiation or field can be an added property of the label, can be a characteristic of the structure of the label, or both. Such radiation or field can also be generated by the combination of the label and some other component, or by a component that is associated with the label, generated by the label, or whose generation is mediated by the label. These latter modes can be referred to as indirect signal generation. Many types of signal, many methods for generating signals, and many methods of detecting signals are known and can be use with and adapted for the disclosed methods and apparatuses. Some signals can be detected visually and these and most other signals can be detected through the use of appropriate instruments. For example, the label in the LA-TCA conjugate can be detected by visual observation of label development at the test zone in the test cell (for labels that produce a visual signal) and/or by the use of an instrument to detect, for example, any radiation or field that may be generated by the label.

In some embodiments of the disclosed method, a liquid sample (which can also be referred to as a test liquid) can be mixed with the labeled agent outside the test cell or prior to bringing the sample into contact with the test cell. In other embodiments, the labeled agent can be disposed in the substrate of the test cell between the inlet and the moderation zone. For example, the labeled agent can be in freeze-dried or other preserved form and/or combined with stabilizing agents on the permeable material between the inlet and the moderation zone. The labeled agent can be disposed in the substrate such that the liquid sample can resolubilize the labeled agent as it passes along the flow path. Methods, techniques and modes for disposition of reagents in test cells and in substrates are known and such methods, techniques and modes can be used to dispose labeled agents in the disclosed test cells.

One or more moderation capture agents can be immobilized in one or more moderation zones of one or more test cells. One or more test capture agents can be immobilized in one or more test zones of one or more test cells. One or more control moderation capture agents can be immobilized in one or more control moderation zones of one or more test cells. One or more control capture agents can be immobilized in one or more control zones of one or more test cells. Moderation capture agents, test capture agents, control moderation capture agents, and control capture agents can be immobilized in any suitable way or manner. Numerous methods and techniques for immobilization of compounds, compositions and reagents are known and can be used for immobilization of moderation capture agents and test capture agents. In particular, techniques known for the immobilization of antibodies, proteins, analytes and oligonucleotides can be used with the disclosed method and test cells. For example, the literature is replete with protein immobilization protocols. See, for example, Laboratory Techniques in Biochemistry and Molecular Biology, Tijssen, Vol. 15, Practice and Theory of Enzyme immunoassays, chapter 13, The Immobilization of Immunoreactants on Solid Phases, pp. 297-328, and the references cited therein. Immobilization as used herein includes binding, coupling, adhering, fixing or other association of the immobilized component to a material. In the context of test cells and substrates of test cells, a component is immobilized when it does not migrate through the test cell with the flow or it migrates only at a rate slower than the rate of the flow. Thus, immobilization can include retardation of movement relative to the flow (and/or relative to other, non-immobilized components). Attachment of assay components to particles, beads or other small structures and embedding the particles, beads or other small structures into the substrate of a test cell, where the particles, beads or other small structures cannot migrate or migrate more slowly than the flow, is an example of a manner of immobilizing assay components.

In forms of the method where moderation capture agents are not immobilized, the moderation capture agents can be removed or separated from the flow by means of binding, capture or sorting. For example, moderation capture agents can be attached to magnetic beads which can be held in place by a magnetic field. As another example, moderation capture agents can include a member of a binding pair, such as biotin or streptavidin, and then captured by the other member of the binding pair immobilized somewhere in the flow path. In these modes, non-immobilized moderation capture agents can be allowed to migrate along the flow until they are removed or separated from the flow (which can be generally before reaching the test zone). This removal accomplishes the same purpose as immobilization: keeping labeled agent bound to moderation capture agents separate from labeled agent bound to test capture agent and from unbound labeled agent. Relevant control components can be set up in a similar configuration.

In some embodiments, the amount or level of signal detected in the test zone can be compared with the amount or level of signal detected in the moderation zone. Depending on the amount of analyte present (and on the relative amount of analyte, labeled agent and moderation capture agent), all, substantially all, some, substantially none, or none of the labeled agent may bind to the moderation capture agent. Thus, the amount of labeled agent that accumulates in the moderation zone depends on the amount of analyte in the sample. Depending on the relative amount of labeled agent and test capture agent, some, substantially all, or all of the labeled agent that does not bind the moderation capture agent will bind the test capture agent. In some embodiments, it is preferred that enough test capture agent be used to bind all of the labeled agent. The amount of labeled agent that accumulates in the test zone depends on the amount of labeled agent that accumulates in the moderation zone, which depends on the amount of analyte in the sample. The ratio of label detected in the moderation zone to label detected in the test zone can provide information about the amount of analyte present in the sample and can serve as an internal control that the expected interactions are taking place in the method.

Label detected in the test zone (and/or moderation zone) can be compared with label of one or more standards or internal controls to determine whether the label detected is a true indication of the presence or absence of the analyte, or an artifact caused by interfering conditions. Control elements in the disclosed method are useful, for example, for detecting conditions in samples or assays that can interfere with the method. For example, high or low pH, the presence of detergent, the presence of cross-linking agents in samples (such glutaraldehyde) can affect or prevent or alter the interactions and bindings for which the method is designed. Thus, in some forms, controls will provide characteristic results when interfering conditions exist in a sample or assay. False positives and analyte specific reactivity of reagents employed can be moderated by the selection of associated test reagents employed including buffers, detergents, flow agents, blocking agents, and stabilizing agents.

One control format generally involves use of one or more control labeled agents (CLA) that can bind to one or more control capture agents (CCA). Binding of the control labeled agent to the control capture agent can serve as an indication that binding can occur in the samples being used. The control labeled agent can comprise a binding agent and a label. The label of the control labeled agent that is bound to control capture agent can be detected. Such detection indicates that binding can occur in the samples being used and can provide assurance that non-detection of analyte in the method is accurate and not due to some interfering condition in the samples.

As used herein, a control labeled agent belongs to the same test set as a control capture agent (and vice versa) when the control capture can bind the control labeled agent, directly or indirectly, in a manner which is typically non-interfering with the test system for the sample analyte. Such groupings of test set components generally can be determined in the context of particular embodiments of the disclosed method and apparatus and need not be based on permanent or absolute relationships between the moderation capture agents, test capture agents, and labeled agents. Control labeled agents and control capture agents can be any type of molecule, compound, or composition. In useful forms of the method, control labeled agents and control capture agents can be chosen such that the type and nature of the interaction of the control labeled agents and control capture agents will be identical, similar or analogous to the type and nature of interactions of one or more labeled agents and the moderation capture agents and/or test capture agents with which they are used thereby comprising a control test set. Such relationships allow the control components to better model the test assay components and their interactions.

The control capture agents can be immobilized in one or more control zones. In forms of the method performed using one or more test cells, the sample zones generally can be upstream of the control zones. For ease of detection, it is useful to dispose control zones in different locations than test zones. However, control zones and test zones also can be partially or fully overlapping. In cases where a control zone fully overlaps with a test zone, it is useful to use labels on the labeled agents and control labeled agents that can be separately detected or whose detection can be distinguished.

Where multiple control zones are used, the zones can have a variety of spatial relationships to each other and to other zones (such as moderation zones and test zones). For example, multiple control zones can be placed sequentially and adjacently along the flow path, with all control zones placed downstream of the moderation zone(s) and/or test zone(s). As another example, control zones can be interspersed, alternated, or both interspersed and alternated with moderation zones and/or test zones. Some useful forms of the disclosed method place all control zones downstream of all moderation zones and all control zones. This configuration exposes the control aspects of the method to relevant portions of the flow path prior to label accumulation in the control zone, thus providing information about the sample and assay conditions through the relevant portions of the flow path.

Control zones can have a variety of geometric configurations. For example, control zones can be lines, bars, open or closed triangles, open or closed rectangles, open or closed squares, open or closed circles, open or closed ellipses, open or closed ovals, open or closed regular polygons, open or closed irregular polygons, open or closed amorphous shapes, spots, rings, cubes, spheres, pyramids, rectangular solids, or any other regular or irregular shape or solid. Control zones need not have clear or distinct boundaries. Thus, for example, control capture agents can be immobilized in a regular or irregular pattern of spots with regular or irregular spaces in between. The area where the control capture agents are immobilized, optionally including or excluding the spaces in between, can be considered a control zone. The same is true of any other disclosed zones.

The disclosed method can use a control labeled agent comprising a binding agent and a label, wherein the label is bound, coupled to or associated with the binding agent. The binding agent of a control labeled agent mediates binding of the control labeled agent to the control capture agent. The binding agent can comprise, for example, a binding region for the control capture agent that belongs to the same test set as the control labeled agent. This binding region for the control capture agent can be referred to as the control moderation binding region.

In some forms of the method, a liquid sample is brought into contact with the control labeled agent. The control labeled agent can then flow along the flow path and be exposed to the same conditions as the other components of the method. Once the control labeled agent reaches the control zone, the control labeled agent can react with the control capture agent of the control zone to form a control labeled agent-control capture agent conjugate (which can be referred to as CLA-CCA conjugate). The CLA-CCA conjugate can be detected in any manner appropriate for the label used. The CLA-CCA conjugate is progressively produced at the control zone as sample continuously passes by. As more and more control labeled agent is immobilized, the label aggregates at the control zone. Detection of the label in the control zone indicates that flow of assay components and intended interactions of assay components can occur under the conditions of the sample and assay.

The label in control labeled agents can be detected by any appropriate means, mode or manner. Generally, the mode or manner of detection can be based on the label used and the type of signal that the label generates. For example, some labels generate or produce radiation or a field that can be detected. Such radiation or field can be an added property of the label, can be a characteristic of the structure of the label, or both. Such radiation or field can also be generated by the combination of the label and some other component, or by a component that is associated with the label, generated by the label, or whose generation is mediated by the label. These latter modes can be referred to as indirect signal generation. Many types of signal, many methods for generating signals, and many methods of detecting signals are known and can be use with and adapted for the disclosed methods and apparatuses. Some signals can be detected visually and these and most other signals can be detected through the use of appropriate instruments. For example, the label in the CLA-CCA conjugate can be detected by visual observation of label development at the test zone in the test cell (for labels that produce a visual signal) and/or by the use of an instrument to detect, for example, any radiation or field that may be generated by the label.

In some embodiments of the disclosed method, a liquid sample can be mixed with the control labeled agent outside the test cell or prior to bringing the sample into contact with the test cell. In other embodiments, the control labeled agent can be disposed in the substrate of the test cell, preferably between the inlet and the moderation zone. For example, the control labeled agent can be in freeze-dried or other preserved form and/or combined with stabilizing agents on the permeable material between the inlet and the moderation zone. The control labeled agent can be disposed in the substrate such that the liquid sample can resolubilize the labeled agent as it passes along the flow path. Methods, techniques and modes for disposition of reagents in test cells and in substrates are known and such methods, techniques and modes can be used to dispose control labeled agents in the disclosed test cells. In useful forms of the method, the liquid sample can be brought into contact with the control labeled agent in the same manner as the liquid sample is brought into contact with the labeled agent. This allows the control labeled agent to be exposed to the same conditions as the labeled agent.

One or more control capture agents can be immobilized in one or more control zones of one or more test cells. Control capture agents can be immobilized in any suitable way or manner. Numerous methods and techniques for immobilization of compounds, compositions and reagents are known and can be used for immobilization of control capture agents. In particular, techniques known for the immobilization of antibodies, proteins, analytes and oligonucleotides can be used with the disclosed method and test cells.

Other control formats can include (along with control labeled agents and control capture agents) one or more control analytes (CA) and/or one or more control moderation capture agents (CMCA). Generally, in these formats, control analytes, control labeled agents, control moderation capture agents and control capture agents can be used in the same ways and with the same relationships as analytes, labeled agents, moderation capture agents, and test capture agents. The purpose of these formats is to further model, in a control format, the interactions of the components of the test assay format. Thus, some control formats can generally involve (1) no binding or reduced binding of control labeled agents to control moderation capture agents in the presence of one or more of control analytes, and (2) binding of control labeled agents that are not bound to control moderation capture agents to one or more control capture agents.

In useful forms of the method, the control moderation capture agents and control capture agents can be immobilized at different physical locations and they can be exposed to the combination of control labeled agents and samples sequentially. For example, a liquid flow of combined sample and control labeled agent can flow through one or more control moderation zones (where one or more control moderation capture agents can be immobilized) and then through one or more control zones (where one or more control capture agents can be immobilized). The control moderation capture agents can be immobilized in one or more moderation zones. The control components in forms of the method involving control analytes and control moderation capture agents can be configured in an analogous manner to either or both the LA format and MCA format. Test set groupings of control analytes, control labeled agents, control moderation capture agents and control capture agents in these formats are analogous to the test set groupings of analytes, labeled agents, moderation capture agents and test capture agents in the test assay formats.

In some embodiments, the amount or level of signal detected in the control zone can be compared with the amount or level of signal detected in the control moderation zone. Depending on the amount of control analyte used (and on the relative amount of control analyte, control labeled agent and control moderation capture agent), all, substantially all, some, substantially none, or none of the control labeled agent may bind to the control moderation capture agent. Thus, the amount of control labeled agent that accumulates in the control moderation zone depends on the amount of control analyte, control labeled agent, and control moderation capture agent used. Depending on the relative amount of control labeled agent and control capture agent, some, substantially all, or all of the control labeled agent that does not bind the control moderation capture agent will bind the control capture agent. In some embodiments, it is preferred that enough control capture agent be used to bind all of the control labeled agent. In some embodiments, it is preferred that enough control moderation capture agent be used to bind only some of the control labeled agent, such as, for example, about half of the control labeled agent. The amount of labeled agent that accumulates in the control zone depends on the amount of control labeled agent that accumulates in the control moderation zone, which depends on the amount of control analyte, control labeled agent, and control moderation capture agent used. The ratio of label detected in the control moderation zone to label detected in the control zone can provide information about the conditions of the sample or assay and thus can serve as a control that the expected interactions are taking place in the method.

In some embodiments, a control zone can be prepared identically to the test zone, except no test capture agent is immobilized in the control zone. In this case, labeled agent that aggregates in the control zone aggregates due only to non-specific binding. If the test zone is not appreciably more intense in label than the control zone, the assay is considered negative.

Useful apparatuses for use with the disclosed method include test cells. Test cells are engineered to comprise a flow path through which liquid samples can flow, for example, through a porous material and/or via channels. Generally the flow path of a test cell can comprise at least one moderation zone and at least one test zone. The test cell and flow path can be constructed of any suitable material. Generally the flow path can comprise a void space, such as channels, and/or a material through which liquid or fluid can flow. The test cell can consist of the flow path or can further comprise other elements and structures. For example, useful test cells comprise a body, which can be unitary or multipart, in which the flow path is disposed. The body can comprise one or more inlets where one or more fluids can be introduced to the test cell. Flow path material can also extend outside the body of the flow cell and such flow path material can be used to introduce fluid to the test cell.

Useful flow paths can comprise a permeable material through which liquid or fluid can flow and/or can comprise impermeable material, such as channels, that directs the flow of the liquid. Impermeable material can comprise plastic, metal, glass, polymer, semiconductor, or other materials useful to channel liquid flow to various zones and chambers. For example, flow paths comprising permeable material can comprise polymers, carbohydrates, fibers, filters, threads, particles, beads, gels, or any combination of these or other permeable materials. Many materials are known for mediating fluid flow and these materials can be used in and adapted for the disclosed test cells. The material for the flow path of a test cell can be selected based on the type of flow (for example, capillary flow, electrophoretic force, gravity (natural or artificial), and chromatographic flow). The flow path can be unitary or multipart. For example, flows paths can comprise a single substantially homogenous material, a single heterogeneous material, multiple different elements of the same materials, multiple different elements of different materials, or any combination. For example, a flow path can comprise a single element. Different components for use in the disclosed method can be disposed in different regions of this single element. As another example, a flow path can comprise a base material in which or on which one or more pads, membranes or pieces are located. In such embodiments, it is useful to provide different components for use in the disclosed method on different pads, membranes or pieces. For example, one or more pads, membranes or pieces can comprise or embody one or more moderation zones, one or more test zones, one or more control zones, one or more control moderation zones, one or more reservoir zones, one or more label zones, and/or one or more sample zones.

An example of a useful test cell can have an elongate outer casing (the body of the test cell) which can house an interior permeable material capable of transporting an aqueous solution through the length of the test cell (the flow path of the test cell). The casing can define a sample inlet, and interior regions which, for ease of description, can be designated as a test volume. The flow path can be disposed in the test volume. The interior region can also comprise an optional reservoir volume. The reservoir zone of the flow path can be disposed in the reservoir volume. If present, the reservoir volume can be disposed in a section of the test cell spaced apart from the inlet. The reservoir zone of the flow path can be, for example, a sorbent material. The reservoir zone can act to receive liquid transported along the flow path defined by the permeable material and extending from the inlet and through the test volume. The body of test cells can be closed save for one or more inlets for the introduction of fluids to the test cell. The body of test cells can also include one or more windows through which observations or measurements can be made of the flow path and components in the flow path. Such windows can be solid, open, transparent, opaque or in other condition. Detection need not require a window. For detection purposes all that is required is that measurement or detection of label be possible. Thus, for example, a visual observation can make use of a transparent or translucent window. A radiation or field detection by an instrument may not require any window if the radiation or field can penetrate the body of the test cell. Alternatively, the window can be made of a material transparent or translucent to the radiation or field to be detected. A window can also be an uncovered opening onto the flow path. A window can also be open or exposed or can be openably covered with some other material. Certain labels can be detected in situ due to the characteristics of the label, such as the electrical properties of semiconductor or metal colloids by measuring changes of conductivity or impedance within the strips at various zones.

The test volume and/or flow path can have a substantially constant cross-sectional shape and/or cross-sectional area. Alternatively, the test volume and/or flow path can include one or more changes in cross-sectional shape and/or cross-sectional area along the test volume and/or flow path. For example, the flow path can be restricted or narrowed in the moderation and test zones, thereby channeling and concentrating fluid flow into contact with the moderation and test zones.

The test cell can also include one or more solution filtering materials (which can also be referred to as filters or filtration materials) disposed in the flow path. Such filters can be disposed anywhere in the flow path as may be useful or appropriate. For example, a filter can be disposed between an inlet and a moderation zone. Filters can be part of the flow path. Filters can comprise separate filter elements (such as a pad) disposed within the body of the test cell in fluid communication with the flow path, but can also be integrated into other materials of the flow path.

In some forms of the disclosed method, using a test cell, the method can be conducted by contacting a sample with the test cell. This can be done in a variety of ways including for example, placing the inlet of the test cell in contact with the sample. One then waits for the test sample to pass through the test cell, where the sample passes into reactive contact with the moderation zone(s) and test zone(s). In some embodiments, the labeled agent can be mixed with the sample and incubated briefly before being contacted with the test cell. In these embodiments, the liquid sample can be contacted with the test cell by placing the inlet of the test cell in contact with the sample or directly adding the liquid sample to the moderation zone of the test cell. In other embodiments, the labeled agent can be disposed in preserved form in the flow path within the test cell. The sample passes through the inlet and the interior of the test cell along the flow path past the moderation, test and control zones, where reactions take place as described elsewhere herein. The labeled agent-test capture agent conjugate that results in the presence of analyte comprises the immobilized test capture agent bound to the labeled agent. The presence of the labeled agent-test capture agent conjugate (and thus the presence of analyte in the sample) can be indicated by detection of label in the test zone.

By providing a reservoir of sorbent material disposed beyond the moderation, test, and control zones, a relatively large volume of liquid sample and any analyte it contains can be drawn through the flow path. Optionally, the region of the flow path in the test cell defining the moderation, test, and control zones can be restricted in cross-sectional area relative to other regions of the flow path. This feature produces a “bottle-neck” effect where all analyte in the entire volume of sample must pass through the restricted flow area immediately about the moderation and test zones.

Where multiple sample zones and/or multiple label zones are used, the various zones can have a variety of spatial relationships. For example, multiple sample zones can be placed sequentially and adjacently along the flow path, with all sample zones placed upstream of the label zone(s). As another example, sample zones can be interspersed, alternated, or both interspersed and alternated with label zones. In such cases, the label zone(s) that belong to the same test set as particular sample zone(s) generally should be placed downstream of the sample zone(s) that belongs to the same test set as the label zone. As used herein, a sample zone belongs to the same test set as a label zone (and vice versa) when the sample zone is used to bring a sample into contact with the flow path where the sample includes or is suspected of containing at least one analyte that belongs to the same test set as at least one labeled agent in the label zone. Thus, for example, sample zones and label zones can be alternated or placed parallel with each sample zone and label zone that belongs to the same test set adjacent to each other.

Where multiple label zones and/or multiple moderation zones are used, the various zones can have a variety of spatial relationships. For example, multiple label zones can be placed sequentially and adjacently along the flow path, with all label zones placed upstream of the moderation zone(s). As another example, label zones can be interspersed, alternated, or both interspersed and alternated with moderation zones. In such cases, the moderation zone(s) that belong to the same test set as particular label zone(s) generally should be placed downstream of the label zone(s) that belongs to the same test set as the moderation zone. As used herein, a label zone belongs to the same test set as a moderation zone (and vice versa) when the label zone includes at least one labeled agent that belongs to the same test set as at least one moderation capture agent in the moderation zone. Thus, for example, label zones and moderation zones can be alternated or placed parallel with each label zone and moderation zone that belongs to the same test set adjacent to each other.

In a similar way, any of the disclosed zones can be placed in a variety of special relationships to any other zones. Generally, zones that belong to the same test set can be placed in the order: sample zone (if present), label zone (if present), moderation zone, test zone, control zone (if present), and reservoir zone (if present). If present, control label zones generally can be placed upstream of the moderation zone and control moderation zone (if present) that belong to the same test set. If present, control moderation zones generally can be placed upstream of the test zone and control zone (if present) that belong to the same test set and downstream of the label zone and the control label zone (if present) that belong to the same test set. If present, control zones generally can be placed downstream of the moderation zone and the test zone that belong to the same test set. As used herein, control label zones, control moderation zones, and control zones belong to the same test set as label zones, moderation zones and test zones if the control components used in the control label zones, control moderation zones, and control zones are intended to serves as controls for the label zones, moderation zones and test zones.

Various zones can also be partially or fully overlapping. For example, label zones and control label zones (belonging to the same test set or not) can be partially or fully overlapping; moderation zones and control moderation zones (belonging to the same test set or not) can be partially of fully overlapping; test zones and control zones (belonging to the same test set or not) can be partially or fully overlapping.

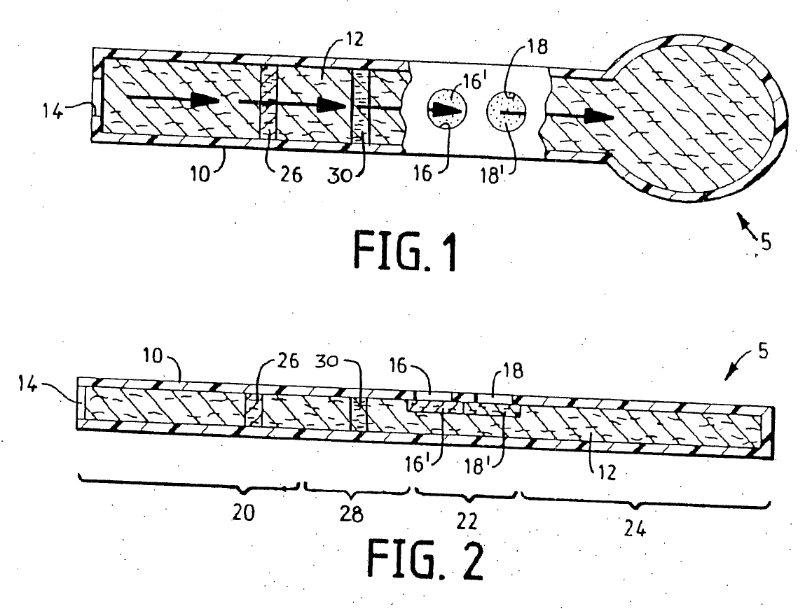

One non-limiting embodiment of the disclosed test cell is schematically illustrated in FIGS. 1 and 2. Test cell 5 can be constructed in accordance with the principles disclosed herein and is useful in explaining its principles of construction. Test cell 5 comprises an outer, molded body or casing 10 which defines a hollow, elongate enclosure (rest volume) that is filled with a substrate 12, which can be a permeable, sorbent material. Substrate 12 comprises the flow path of the test cell. Body 10 also defines an inlet 14 and a pair of circular openings 16, 18 comprising windows through which substrate 12 is visible.

Substrate 12 and the interior of body 10 together define a flow path passing generally from left to right in FIGS. 1 and 2. When the test cell is placed with inlet 14 disposed within or otherwise in contact with a liquid sample, the liquid is transported by capillary action, wicking, or simple wetting along the flow path through downstream flow section 20, moderation volume 28, test volume 22, and into reservoir volume 24, generally as depicted by the arrows.

Disposed within substrate 12 is a band 26 (label zone) which can comprise labeled agent and control labeled agent. As the liquid sample moves past band 26, the labeled agent and control labeled agent are entrained in the liquid, reconstituted. In MCA format test cells, the labeled agent reacts with analyte if present in the liquid sample. Alternatively, the labeled agent and control labeled agent could be disposed in separate bands disposed within the substrate 12. Of course, labeled agent band 26 can be eliminated, and the labeled agent and control labeled agent can be added to the test liquid prior to introduction of the cell 5 as previously noted.

Within the volume of substrate 12 disposed downstream of the flow section 20 of the flow path and upstream of test volume 22, is disposed moderation volume 28 comprising moderation zone 30. Moderation zone 30 comprises a pre-selected quantity of moderation capture agent immobilized in place within the flow path. Also within the volume of substrate 12 disposed directly beneath circular openings 16 and 18 in body 10 is disposed, respectively, test zone 16′ and control zone 18′. In the drawing, the control and test zones are illustrated as being disposed serially along the flow path. Alternatively, the control and test zones or zones can be disposed side by side, in a different order, or in other spatial relationships.

Test zone 16′ comprises a pre-selected quantity of test capture agent immobilized in place within the flow path. Control zone 18′ a pre-selected quantity of control capture agent immobilized in place within the flow path. Thus, aggregation of the labeled agent which can occur at test zone 16′ will not occur at control zone 18′. Only if the control analyte is present will aggregation of the label occur at control zone 18′ occur. Presence of a label at test zone 16′ is a positive indication of the analyte in the sample. Presence of a label at control zone 18′ and the absence of label at test zone 16′ is an indication that no analyte is in the sample or that the level of analyte in the sample is below the level of detection.

The test cell is not limited by test zone 16′ and control zone 18′ depicted in FIGS. 1 and 2. In fact, control zone 18′ can be entirely eliminated. Generally, test capture agent can be immobilized at test zone 16′ using adsorption, absorption, or ionic or covalent coupling, in accordance with methods known. In some embodiments, test capture agent can be striped onto the test zone or disposed in a pad that can be placed on or within the flow path. In such a case, the pad can be considered the test zone.

Disposed beyond test volume 22 is reservoir volume 24 comprising a relatively large mass of sorbent or supersorbent material (reservoir zone). The purpose of reservoir volume 24 is to assure that a reasonably large amount of liquid is drawn through the moderation volume 28 and the test volume 22. Increasing the volume of reservoir 24 can have the effect of increasing the sensitivity of the assay procedure, as it results in an increase in the amount of analyte passing through the moderation volume 28 and test volume 22. Suitable sorbents include commercial materials of the type available, for example, from The Dow Chemical Company of Midland, Mich., and the Chemical division of American Colloid, Arlington Heights, Ill. These materials can absorb many times their weight in water and are commonly used in disposable diapers. They can comprise lightly cross-linked polyacrylate salts, typically alkali metal salts, cellulose, desiccant material such as silica gel. The sorbent material can be molded into housing 10 to comprise reservoir volume 24 by contact with test volume 22.

FIG. 3 depicts an example of the disclosed method and device in the LA format. The test cell (test strip) body is a surface which serves as an impermeable backing for the flow path. The sample pad is part of the flow path and includes a sample zone (where the sample is introduced), a label zone (where the labeled agent is detachably immobilized), and a control label zone (where the control labeled agent is detachably immobilized). The labeled agent in this example competes with the analyte for binding to the moderation capture agent. The control labeled agent in this example is the labeled peptide. The moderation zone, on the main part of the flow path (nitrocellulose in this example), is where the moderation capture agent (in this example, anti-analyte analogue antibody) is immobilized. An optional control moderation zone, where control moderation capture agent (in this example, anti-peptide antibody) is immobilized, is downstream on the main part of the flow path. Downstream from the moderation zones on the main part of the flow path is a test zone (test line) where test capture agent (in this example, anti-antibody antibody) is immobilized. Downstream of the test zone on the main part of the flow path is a control zone where control capture agent (in this example, anti-peptide antibody) is immobilized. Downstream from the control zone is an absorbent pad, also part of the flow path, which constitutes a reservoir zone.

FIG. 4 depicts an example of the disclosed method and device in the MCA format. The test cell (test strip) body is an impermeable surface which serves as a backing for the flow path. The sample pad is part of the flow path and includes a sample zone (where the sample is introduced), a label zone (where the labeled agent is detachably immobilized), and a control label zone (where the control labeled agent is detachably immobilized). The labeled agent in this example is the labeled anti-analyte antibody. The control labeled agent in this example is the labeled peptide. The moderation zone, on the main part of the flow path (nitrocellulose in this example), is where the moderation capture agent (in this example, analyte analogue) is immobilized. An optional control moderation zone, where control moderation capture agent (in this example, anti-peptide antibody) is immobilized, is downstream on the main part of the flow path. Downstream from the moderation zones on the main part of the flow path is a test zone (test line) where test capture agent (in this example, anti-antibody antibody) is immobilized. Downstream of the test zone on the main part of the flow path is a control zone where control capture agent (in this example, anti-peptide antibody) is immobilized. Downstream from the control zone is an absorbent pad, also part of the flow path, which constitutes a reservoir zone.

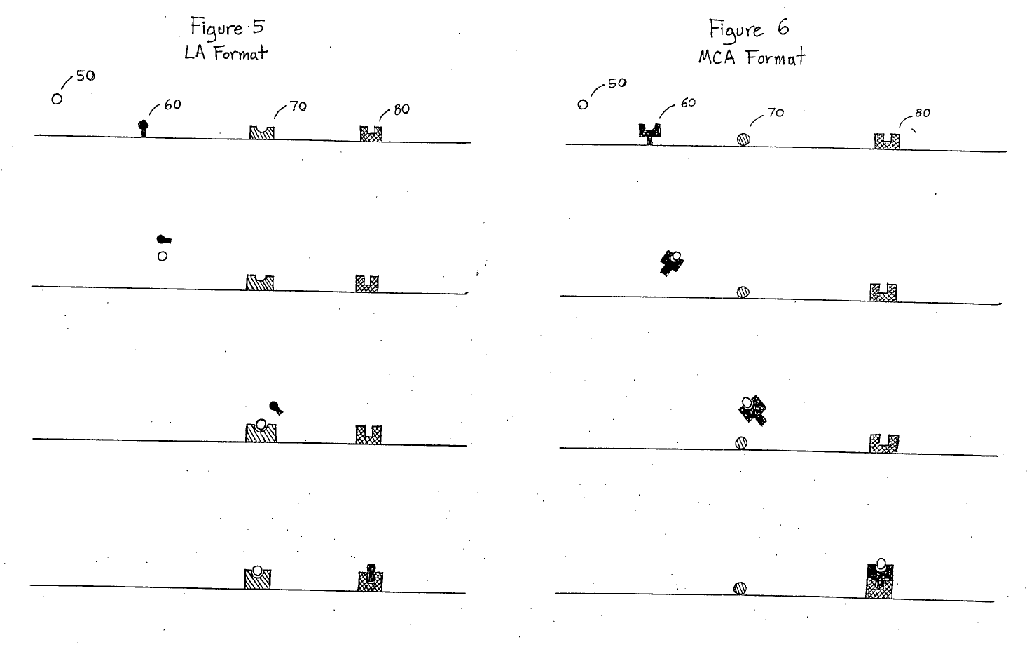

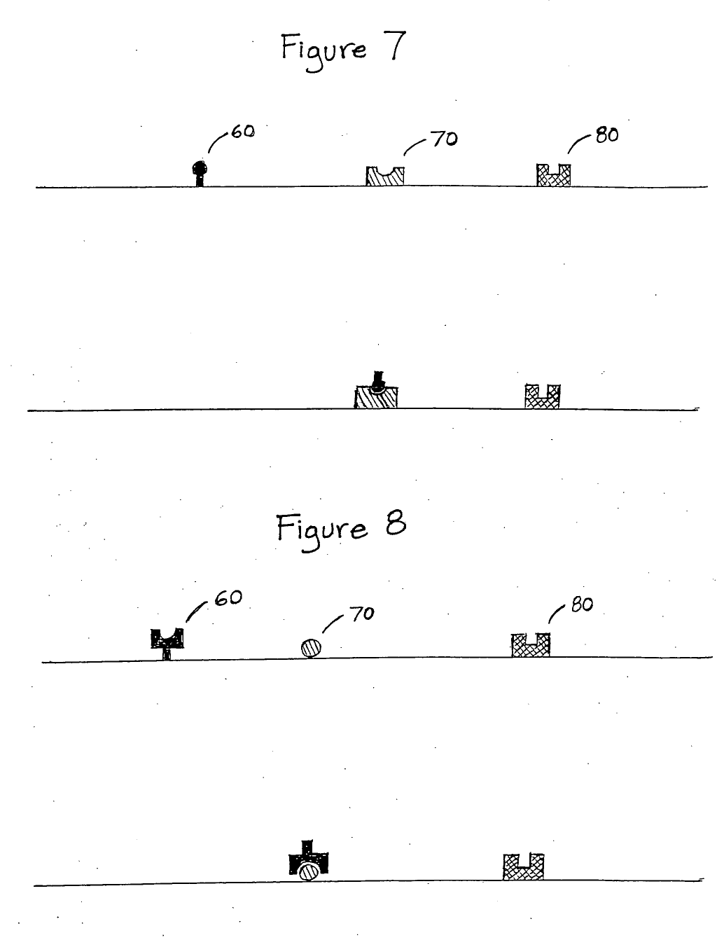

FIG. 5 depicts an example of the LA format of disclosed method. In the top panel, analyte 50 flows toward detachably localized labeled agent 60, immobilized moderation capture agent 70 and immobilized test capture agent 80. In the second panel, analyte 50 and the sample have reached labeled agent 60, and labeled agent 60 has been released to flow with the sample. In the third panel, analyte 50 and labeled agent 60 have reached moderation capture agent 70. Analyte 50 and labeled agent 60 compete with each other for binding to moderation capture agent 70, with the result, in this example, that analyte 50 binds to moderation capture agent 70 and prevents labeled agent 60 from binding moderation capture agent 70. In the last panel, labeled agent 60, having not bound moderation capture agent 70, has reached test capture agent 80 and bound to test capture agent 80. The label of labeled agent 60 would be detectable at the site of immobilization of test capture agent 80. FIG. 5 shows the case when analyte is present in the sample. FIG. 7 depicts an example of the LA format of the disclosed method when analyte is not present in the sample. The top panel shows detachably localized labeled agent 60, immobilized moderation capture agent 70 and immobilized test capture agent 80. In the second panel, labeled agent 60 has detached and flowed with the sample to moderation capture agent 70. Because no analyte is present to compete with labeled agent 60 for binding to moderation capture agent 70, labeled agent 60 binds to moderation capture agent 70. Little or no labeled agent 60 flows to and binds to test capture agent 80. As a result, the label of labeled agent 60 would not be readily detectable at the site of immobilization of test capture agent 80.

FIG. 6 depicts an example of the MCA format of disclosed method. In the top panel, analyte 50 flows toward detachably localized labeled agent 60, immobilized moderation capture agent 70 and immobilized test capture agent 80. In the second panel, analyte 50 and the sample have reached labeled agent 60, and labeled agent 60 has been released to flow with the sample and binds to the analyte. Labeled agent 70 binds to analyte 50. In the third panel, analyte 50 and labeled agent 60 have reached moderation capture agent 70. Analyte 50 and moderation capture agent 70 compete with each other for binding to labeled agent 60, with the result, in this example, that analyte 50 remains bound to labeled agent 60 and prevents analyte 50 from binding moderation capture agent 70. In the last panel, labeled agent 60 (and analyte 50), having not bound moderation capture agent 70, has reached test capture agent 80 and bound to test capture agent 80. The label of labeled agent 60 would be detectable at the site of immobilization of test capture agent 80. FIG. 6 shows the case when analyte is present in the sample. FIG. 8 depicts an example of the MCA format of the disclosed method when analyte is not present in the sample. The top panel shows detachably localized labeled agent 60, immobilized moderation capture agent 70 and immobilized test capture agent 80. In the second panel, labeled agent 60 has detached and flowed with the sample to moderation capture agent 70. Because no analyte is present to compete with moderation capture agent 70 for binding to labeled agent 60, labeled agent 60 binds to moderation capture agent 70. Little or no labeled agent 60 flows to and binds to test capture agent 80. As a result, the label of labeled agent 60 would not be readily detectable at the site of immobilization of test capture agent 80.

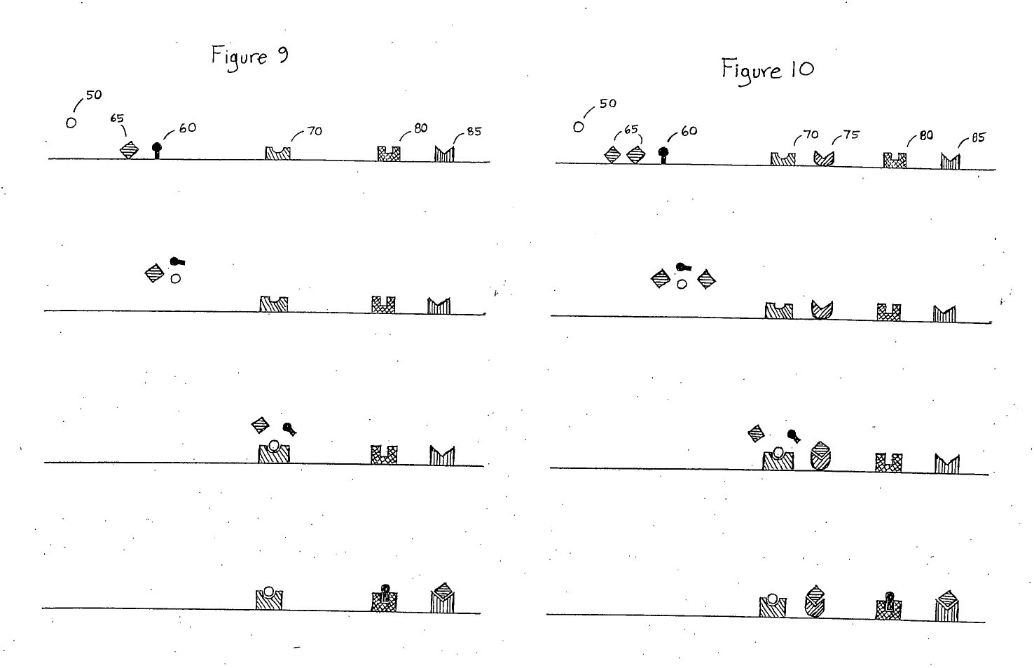

FIG. 9 depicts an example of the LA format of disclosed method that includes a control. In the top panel, analyte 50 flows toward detachably localized control labeled agent 65, detachably localized labeled agent 60, immobilized moderation capture agent 70, immobilized test capture agent 80, and immobilized control capture agent 85. In the second panel, analyte 50 and the sample have reached control labeled agent 65 and labeled agent 60, and control labeled agent 65 and labeled agent 60 have been released to flow with the sample. In the third panel, analyte 50, control labeled agent 65, and labeled agent 60 have reached moderation capture agent 70. Analyte 50 and labeled agent 60 compete with each other for binding to moderation capture agent 70, with the result, in this example, that analyte 50 binds to moderation capture agent 70 and prevents labeled agent 60 from binding moderation capture agent 70. In the last panel, labeled agent 60, having not bound moderation capture agent 70, has reached test capture agent 80 and bound to test capture agent 80. Control labeled agent 65 has reached control capture agent 85 and bound to control capture agent 85. The label of labeled agent 60 would be detectable at the site of immobilization of test capture agent 80. The label of control labeled agent 65 would be detectable at the site of immobilization of test capture agent 80.

FIG. 10 depicts an example of the LA format of disclosed method that includes a control. In the top panel, analyte 50 flows toward detachably localized control labeled agent 65, detachably localized labeled agent 60, immobilized moderation capture agent 70, immobilized control moderation capture agent 75, immobilized test capture agent 80, and immobilized control capture agent 85. In the second panel, analyte 50 and the sample have reached control labeled agent 65 and labeled agent 60, and control labeled agent 65 and labeled agent 60 have been released to flow with the sample. In the third panel, analyte 50, control labeled agent 65, and labeled agent 60 have reached moderation capture agent 70 and control moderation capture agent 75. Analyte 50 and labeled agent 60 compete with each other for binding to moderation capture agent 70, with the result, in this example, that analyte 50 binds to moderation capture agent 70 and prevents labeled agent 60 from binding moderation capture agent 70. Some of control labeled agent 65 binds to control moderation capture agent 75. In the last panel, labeled agent 60, having not bound moderation capture agent 70, has reached test capture agent 80 and bound to test capture agent 80. Some of control labeled agent 65 has reached control capture agent 85 and bound to control capture agent 85. The label of labeled agent 60 would be detectable at the site of immobilization of test capture agent 80. The label of control labeled agent 65 would be detectable at the site of immobilization of control moderation capture agent 75 and at the site of immobilization of test capture agent 80.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### Materials

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a labeled agent is disclosed and discussed and a number of modifications that can be made to a number of molecules including the labeled agent are discussed, each and every combination and permutation of labeled agent and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

A. Analytes

An analyte can be an analyte of any nature that one seeks to determine the presence or absence of. Virtually any analyte in any sample can be detected using the disclosed method and apparatus. For example, proteins, peptides, antibodies, small molecules, metabolites, hormones, breakdown products, antigens, epitopes, nucleic acids, lipids, carbohydrates, sugars, cells, spores, viruses, bacteria, inorganic compounds, organic compounds, drugs, poisons, contaminants, and the like are analytes that can be detected with the disclosed methods and apparatuses. The disclosed methods and apparatuses are particularly useful for detecting analytes in, for example, in a liquid sample such as a biological or environmental sample, but the analyte can have any source.

In general, any compound, moiety, or component of a compound or complex can be an analyte. Preferred analytes are peptides, proteins, and other macromolecules such as lipids, complex carbohydrates, proteolipids, membrane fragments, cells, spores, viruses, bacteria, and nucleic acids. Analytes can also be smaller molecules such as cofactors, metabolites, enzyme substrates, metal ions, and metal chelates. Analytes can, but need not, range in size from 100 daltons to 1,000,000 Daltons, but could include whole cells, bacteria, viruses, and spores. It is to be understood that the term analyte refers to both separate molecules and to portions of molecules, such as an epitope of a protein.

Analytes may contain modifications, both naturally occurring or induced in vitro or in vivo. Induced modifications include adduct formation such as hapten attachment, multimerization, complex formation by interaction with other chemical moieties, digestion or cleavage (by, for example, protease), and metal ion attachment or removal. The disclosed method can be used to detect differences in the modification state of an analyte, such as the phosphorylation or glycosylation state of proteins. The method disclosed herein can be used to detect analytes of interest of all sizes, for example, hCG, strep A/B, antibodies to HIV and antibodies to other infectious diseases.

B. Samples

Samples can be derived from any source that has, or is suspected of having, an analyte. Samples can contain particular analytes or a pool of analytes. Samples can include natural molecules, chemically synthesized molecules, or both. A sample can be, for example, a sample from one or more cells, tissue, or bodily fluids such as blood, urine, semen, lymphatic fluid, cerebrospinal fluid, or amniotic fluid, or other biological samples, such as tissue culture cells, buccal swabs, mouthwash, stool, tissues slices, biopsy aspiration, environmental samples such as river, ocean, sewer, swab, wipe, filter (air, water or fluid), soil, rock, metal, ceramic, glass, fabric, chemical, weapons samples such as samples of chemical or biological weapons, and archeological samples such as bone or mummified tissue. Types of useful target samples include blood samples, urine samples, semen samples, lymphatic fluid samples, cerebrospinal fluid samples, amniotic fluid samples, biopsy samples, needle aspiration biopsy samples, cancer samples, tumor samples, tissue samples, cell samples, cell lysate samples, crude cell lysate samples, forensic samples, environmental samples, archeological samples, infection samples, nosocomial infection samples, production samples, drug preparation samples, drug screening samples, chemical samples, chemical weapons samples, bioweapons samples, river samples, ocean samples, sewer samples, swab samples, wipe samples, filter samples (air, water or fluid), soil samples, rock samples, metal samples, ceramic samples, glass samples, fabric samples, biological molecule production samples, protein preparation samples, lipid preparation samples, and/or carbohydrate preparation samples.

C. Labeled Agents (LA)

A labeled agent (LA) can comprise binding agent and a label. A labeled agent requires only a detectable label and the ability to bind or be bound, directly or indirectly, by a moderation capture agent and a test capture agent (and an analyte in the MCA format of the disclosed method). The label can be bound, coupled to or associated with the binding agent directly or indirectly. The binding agent of a labeled agent can mediate binding of the labeled agent to the moderation capture agent (and the analyte in the MCA format). The binding agent can comprise, for example, a binding region for the moderation capture agent that belongs to the same test set as the labeled agent. This binding region for the moderation capture agent can be referred to as the moderation binding region. For use in an MCA format, the binding region generally can be for the analyte.

The labeled agent also can comprise a binding region for the test capture agent. This binding region for the test capture agent (which can be referred to as the test binding region) can be any part or portion of the labeled agent and need not be (but can be or can overlap with) the moderation binding region. The test binding region need not be part of the binding agent of the labeled agent. The test binding region can be a component of the labeled agent present for that purpose. Such a component comprising the test binding region can be referred to as a test binding agent. A test binding agent can be, for example, a marker reagent, such as the blocking agent used in making the labeled agent, an oligonucleotide, or any other component that can be specifically bound. Alternatively, in the MCA format where an analyte-labeled agent conjugate forms, the labeled agent can bind to the test capture agent indirectly via the analyte. Where the test capture agent binds to the analyte in the analyte-labeled agent conjugate, a “sandwich” of the test capture agent, analyte, and the labeled agent can form. The test capture agent can also bind to a structure that is a combination part of the analyte and part of the labeled agent.

The label of the labeled agent that is bound to test capture agent can be detected. Such detection can be used to indicate the presence of one or more of the analytes in one or more of the samples because labeled agents become available to bind test capture agents when the labeled agents fail to bind moderation capture agents in the presence of the analytes.

In the MCA format, analyte and moderation capture agent compete with each other for binding to the labeled agent. In the LA format, analyte and labeled agents compete with each other for binding to the moderation capture agent. Competition for binding can be accomplished by including molecules, elements or moieties in the labeled agents that are similar or identical to molecules, elements or moieties of the analyte that belongs to the same test set as the labeled agent. In the LA format, useful binding agents in the labeled agent can be analyte that belongs to the same test set, an analyte mimic or mimetic of the analyte that belongs to the same test set, or a derivative of the analyte that belongs to the same test set.

As used herein, a labeled agent belongs to the same test set as a moderation capture agent (and vice versa) when the moderation capture agent can bind the labeled agent in the absence of the analyte. As used herein, an analyte belongs to the same test set as a labeled agent and a moderation capture agent (and vice versa) when the analyte interferes with the binding of the labeled agent to the moderation capture agent by competing with the labeled agent for binding to the moderation capture agent or by competing with the moderation capture agent for binding to the labeled agent. As used herein, a labeled agent belongs to the same test set as a test capture agent (and vice versa) when the test capture agent can bind the labeled agent, directly or indirectly. Such groupings of test set components generally can be determined in the context of particular embodiments of the disclosed method and apparatus and need not be based on permanent or absolute relationships between the moderation capture agents, test capture agents, and labeled agents.

In some embodiments, of the disclosed method and apparatus, the sample can be mixed with the labeled agent outside the test cell. In other embodiments, the labeled agent can be disposed in freeze-dried or other preserved form and/or combined with stabilizing agents on permeable material between the inlet and the moderation zone. The liquid sample can then resolubilize the labeled agent as the liquid sample passes along the flow path.

Sets or groups of labeled agents can be used together in the claimed method. Different labeled agents can be distinguished or separately detected by using different labels for different labeled agents or sets of labeled agents, by having the labeled agents in the same test sets as different test capture agents or different sets of test capture agents immobilized in different locations, or a combination of these strategies. For example, different labeled agents can have different test binding regions, each specific for particular test capture agents. As a specific example of this, different labeled agents can have different oligonucleotides as test binding regions and the test capture agents that belong to the same test set as the labeled agents can comprise complementary oligonucleotides. Such a scheme can provide a high level of multiplexing in the disclosed method.

D. Moderation Capture Agent (MCA)

A moderation capture agent (MCA) can be any molecule, compound, composition or other agent that can bind a labeled agent and either can compete with an analyte for binding to the labeled agent (in the MCA format) or can bind to an analyte (in the LA format). The binding agent of a labeled agent mediates binding of the labeled agent to the moderation capture agent. In moderation capture agents for use in the LA format, the moderation capture agent can bind both a labeled agent and the analyte, with the labeled agent and the analyte competing for binding to the moderation capture agent.

In the MCA format, an analyte competes with a moderation capture agent for binding to a labeled agent. Competition for binding can be accomplished by including molecules, elements or moieties in the moderation capture agent that are similar or identical to molecules, elements or moieties of the analyte that belongs to the same test set as the moderation capture agent. In the MCA format, useful moderation capture agents can be the analyte that belongs to the same test set, an analyte mimic or mimetic of the analyte that belongs to the same test set, or a derivative of the analyte that belongs to the same test set.

Moderation capture agents can be immobilized at different physical locations. Moderation capture agents can be immobilized in one or more moderation zones. The various zones can have a variety of spatial relationships. For example, multiple moderation zones can be placed sequentially and adjacently along the flow path, with all moderation zones placed upstream of the test zone(s). As another example, moderation zones can be interspersed, alternated, or both interspersed and alternated with test zones. In such cases, the test zone(s) belong to the same test set as particular moderation zone(s) generally should be placed downstream of the moderation zone(s) that belongs to the same test set.

Moderation zones can have a variety of geometric configurations. For example, moderation zones can be lines, bars, open or closed triangles, open or closed rectangles, open or closed squares, open or closed circles, open or closed ellipses, open or closed ovals, open or closed regular polygons, open or closed irregular polygons, open or closed amorphous shapes, spots, rings, cubes, spheres, pyramids, rectangular solids, or any other regular or irregular shape or solid. Moderation zones need not have clear or distinct boundaries. Thus, for example, moderation capture agents can be immobilized in a regular or irregular pattern of spots with regular or irregular spaces in between. The area where the moderation capture agents are immobilized, optionally including or excluding the spaces in between, can be considered a moderation zone.

Moderation capture agents can be immobilized in any suitable way or manner. Numerous methods and techniques for immobilization of compounds, compositions and reagents are known and can be used for immobilization of moderation capture agents. In particular, techniques known for the immobilization of antibodies, proteins, analytes and oligonucleotides can be used with the disclosed method and test cells. For example, the literature is replete with protein immobilization protocols. See, for example, Laboratory Techniques in Biochemistry and Molecular Biology, Tijssen, Vol. 15, Practice and Theory of Enzyme immunoassays, chapter 13, The Immobilization of Immunoreactants on Solid Phases, pp. 297-328, and the references cited therein.

As used herein, a labeled agent belongs to the same test set as a moderation capture agent (and vice versa) when the moderation capture agent can bind the labeled agent in the absence of the analyte. As used herein, an analyte belongs to the same test set as a labeled agent and a moderation capture agent (and vice versa) when the analyte interferes with the binding of the labeled agent to the moderation capture agent by competing with the labeled agent for binding to the moderation capture agent or by competing with the moderation capture agent for binding to the labeled agent. As used herein, a moderation zone belongs to the same test set as a test zone (and vice versa) when the moderation zone includes at least one moderation capture agent that belongs to the same test set as at least one test capture agent in the test zone. As used herein, a moderation capture agent belongs to the same test set as a test capture agent (and vice versa) when the moderation capture agent and test capture agent can both bind the same labeled agent, directly or indirectly. Such groupings of test set components generally can be determined in the context of particular embodiments of the disclosed method and apparatus and need not be based on permanent or absolute relationships between the moderation capture agents, test capture agents, and labeled agents.

E. Test Capture Agent (TCA)

A test capture agent (TCA) can be any molecule, compound, composition or other agent that can bind a labeled agent, directly or indirectly. The test binding region or agent of a labeled agent mediates binding of the labeled agent to the test capture agent. Test capture agents can be immobilized at different physical locations. Test capture agents can be immobilized in one or more test zones. The various zones can have a variety of spatial relationships. For example, multiple test zones can be placed sequentially and adjacently along the flow path, with all test zones placed downstream of the moderation zone(s). As another example, test zones can be interspersed, alternated, or both interspersed and alternated with moderation zones. In such cases, the test zone(s) that belongs to the same test set as particular moderation zone(s) generally should be placed downstream of the moderation zone(s) that belong to the same test set.

Test zones can have a variety of geometric configurations. For example, test zones can be lines, bars, open or closed triangles, open or closed rectangles, open or closed squares, open or closed circles, open or closed ellipses, open or closed ovals, open or closed regular polygons, open or closed irregular polygons, open or closed amorphous shapes, spots, rings, cubes, spheres, pyramids, rectangular solids, or any other regular or irregular shape or solid. Test zones need not have clear or distinct boundaries. Thus, for example, test capture agents can be immobilized in a regular or irregular pattern of spots with regular or irregular spaces in between. The area where the test capture agents are immobilized, optionally including or excluding the spaces in between, can be considered a test zone.

Test capture agents can be immobilized in any suitable way or manner. Numerous methods and techniques for immobilization of compounds, compositions and reagents are known and can be used for immobilization of test capture agents. In particular, techniques known for the immobilization of antibodies, proteins, analytes and oligonucleotides can be used with the disclosed method and test cells. For example, the literature is replete with protein immobilization protocols. See, for example, Laboratory Techniques in Biochemistry and Molecular Biology, Tijssen, Vol. 15, Practice and Theory of Enzyme immunoassays, chapter 13, The Immobilization of Immunoreactants on Solid Phases, pp. 297-328, and the references cited therein.

As used herein, a labeled agent belongs to the same test set as a test capture agent (and vice versa) when the test capture agent can bind the labeled agent. As used herein, a moderation capture agent belongs to the same test set as a test capture agent (and vice versa) when the moderation capture agent and test capture agent can both bind the same labeled agent, directly or indirectly. As used herein, a moderation zone belongs to the same test set as a test zone (and vice versa) when the moderation zone includes at least one moderation capture agent that belongs to the same test set as at least one test capture agent in the test zone. Such groupings of test set components generally can be determined in the context of particular embodiments of the disclosed method and apparatus and need not be based on permanent or absolute relationships between the moderation capture agents, test capture agents, and labeled agents.

F. Control Labeled Agent (CLA)

A control labeled agent (LA) comprises a binding agent and a label. A control labeled agent requires a detectable label and the ability to bind or be bound by a control capture agent. The label can be bound, coupled to or associated with the binding agent. The binding agent of a control labeled agent can mediate binding of the labeled agent to the control capture agent. The binding agent can comprise, for example, a binding region for the control capture agent that belongs to the same test set as the control labeled agent. This binding region for the control capture agent can be referred to as the control binding region.

The labeled agent also can comprise a binding region for the control moderation capture agent. This binding region for the control moderation capture agent (which can be referred to as the control moderation binding region) can be any part or portion of the control labeled agent and need not be (but can be or can overlap with) the control binding region. The test binding region need not be part of the binding agent of the labeled agent. The label of the control labeled agent that is bound to control capture agent can be detected.

As used herein, a control labeled agent belongs to the same test set as a control capture agent (and vice versa) when the control capture agent can bind the control labeled agent. As used herein, a control analyte belongs to the same test set as a control labeled agent and a control moderation capture agent (and vice versa) when the analyte interferes with the binding of the control labeled agent to the control moderation capture agent by competing with the control labeled agent for binding to the control moderation capture agent or by competing with the control moderation capture agent for binding to the control labeled agent. Such groupings of test set components generally can be determined in the context of particular embodiments of the disclosed method and apparatus and need not be based on permanent or absolute relationships between the control moderation capture agents, control capture agents, and control labeled agents. Typically, the control test set (control label agent, control capture agent, and control analyte) are selected so that they do not cross react or otherwise interfere with the sample analyte, or the sample analyte test components (LA, MCA and TCA).

In some embodiments of the disclosed method and apparatus, the sample can be mixed with the control labeled agent outside the test cell. In other embodiments, the control labeled agent can be disposed in freeze-dried or other preserved form and/or combined with stabilizing agents on permeable material between, for example, the inlet and the moderation zone. The liquid sample can then resolubilize the control labeled agent as the liquid sample passes along the flow path.

G. Control Capture Agent (CCA)

A control capture agent (CCA) can be any molecule, compound, composition or other agent that can bind a control labeled agent, directly or indirectly. The control binding region or agent of a control labeled agent mediates binding of the control labeled agent to the control capture agent. Control capture agents can be immobilized at different physical locations. Control capture agents can be immobilized in one or more control zones. The various zones can have a variety of spatial relationships. For example, multiple control zones can be placed sequentially and adjacently along the flow path, with all control zones placed downstream of the moderation and test zone. As another example, control zones can be interspersed, alternated, or both interspersed and alternated with moderation and test zones. In such cases, the control zone(s) that belong to the same test set as particular moderation zone(s) and particular test zone(s) generally can be placed downstream of the moderation and test zones that belongs to the same test set.

Control zones can have a variety of geometric configurations. For example, control zones can be lines, bars, open or closed triangles, open or closed rectangles, open or closed squares, open or closed circles, open or closed ellipses, open or closed ovals, open or closed regular polygons, open or closed irregular polygons, open or closed amorphous shapes, spots, rings, cubes, spheres, pyramids, rectangular solids, or any other regular or irregular shape or solid. Control zones need not have clear or distinct boundaries. Thus, for example, control capture agents can be immobilized in a regular or irregular pattern of spots with regular or irregular spaces in between. The area where the control capture agents are immobilized, optionally including or excluding the spaces in between, can be considered a control zone.

Control capture agents can be immobilized in any suitable way or manner. Numerous methods and techniques for immobilization of compounds, compositions and reagents are known and can be used for immobilization of control capture agents. In particular, techniques known for the immobilization of antibodies, proteins, analytes and oligonucleotides can be used with the disclosed method and test cells. For example, the literature is replete with protein immobilization protocols. See, for example, Laboratory Techniques in Biochemistry and Molecular Biology, Tijssen, Vol. 15, Practice and Theory of Enzyme immunoassays, chapter 13, The Immobilization of Immunoreactants on Solid Phases, pp. 297-328, and the references cited therein.

As used herein, a control labeled agent belongs to the same test set as a control capture agent (and vice versa) when the control capture agent can bind the control labeled agent, directly or indirectly. As used herein, a control moderation capture agent belongs to the same test set as a control capture agent (and vice versa) when the control moderation capture agent and control capture agent can both bind the same control labeled agent, directly or indirectly. As used herein, a control moderation zone belongs to the same test set as a control zone (and vice versa) when the control moderation zone includes at least one control moderation capture agent that belongs to the same test set as at least one control capture agent in the control zone. Such groupings of test set components generally can be determined in the context of particular embodiments of the disclosed method and apparatus and need not be based on permanent or absolute relationships between the control moderation capture agents, control capture agents, and control labeled agents.

H. Control Moderation Capture Agent (CMCA)

A control moderation capture agent (CMCA) can be any molecule, compound, composition or other agent that can bind a control labeled agent and either can compete with a control analyte for binding to the control labeled agent (in the control MCA format) or can bind to a control analyte (in the control LA format). The binding agent of a control labeled agent mediates binding of the control labeled agent to the control moderation capture agent. In control moderation capture agents for use in the control LA format, the control moderation capture agent can bind both a control labeled agent and the control analyte, with the control labeled agent and the control analyte competing for binding to the control moderation capture agent.

In the control MCA format, a control analyte competes with a control moderation capture agent for binding to a control labeled agent. Competition for binding can be accomplished by including molecules, elements or moieties in the control moderation capture agent that are similar or identical to molecules, elements or moieties of the control analyte that belongs to the same test set as the control moderation capture agent. In the control MCA format, useful control moderation capture agents can be the control analyte that belongs to the same test set, a control analyte mimic or mimetic of the control analyte that belongs to the same test set, or a derivative of the control analyte that belongs to the same test set.

Control moderation capture agents can be immobilized at different physical locations. Control moderation capture agents can be immobilized in one or more control moderation zones. The various zones can have a variety of spatial relationships. For example, multiple control moderation zones can be placed sequentially and adjacently along the flow path, with all control moderation zones placed upstream of the control zone(s). As another example, control moderation zones can be interspersed, alternated, or both interspersed and alternated with control zones. In such cases, the control zone(s) that belong to the same test set as particular control moderation zone(s) generally should be placed downstream of the control moderation zone(s) that belongs to the same test set.

Control moderation zones can have a variety of geometric configurations. For example, control moderation zones can be lines, bars, open or closed triangles, open or closed rectangles, open or closed squares, open or closed circles, open or closed ellipses, open or closed ovals, open or closed regular polygons, open or closed irregular polygons, open or closed amorphous shapes, spots, rings, cubes, spheres, pyramids, rectangular solids, or any other regular or irregular shape or solid. Control moderation zones need not have clear or distinct boundaries. Thus, for example, control moderation capture agents can be immobilized in a regular or irregular pattern of spots with regular or irregular spaces in between. The area where the control moderation capture agents are immobilized, optionally including or excluding the spaces in between, can be considered a control moderation zone.

Control moderation capture agents can be immobilized in any suitable way or manner. Numerous methods and techniques for immobilization of compounds, compositions and reagents are known and can be used for immobilization of control moderation capture agents. In particular, techniques known for the immobilization of antibodies, proteins, analytes and oligonucleotides can be used with the disclosed method and test cells. For example, the literature is replete with protein immobilization protocols. See, for example, Laboratory Techniques in Biochemistry and Molecular Biology, Tijssen, Vol. 15, Practice and Theory of Enzyme immunoassays, chapter 13, The Immobilization of Immunoreactants on Solid Phases, pp. 297-328, and the references cited therein.

As used herein, a control labeled agent belongs to the same test set as a control moderation capture agent (and vice versa) when the control moderation capture agent can bind the control labeled agent in the absence of the control analyte. As used herein, a control analyte belongs to the same test set as a control labeled agent and a control moderation capture agent (and vice versa) when the control analyte interferes with the binding of the control labeled agent to the control moderation capture agent by competing with the control labeled agent for binding to the control moderation capture agent or by competing with the control moderation capture agent for binding to the control labeled agent. As used herein, a control moderation zone belongs to the same test set as a control zone (and vice versa) when the control moderation zone includes at least one control moderation capture agent that belongs to the same test set as at least one control capture agent in the control zone. As used herein, a control moderation capture agent belongs to the same test set as a control capture agent (and vice versa) when the control moderation capture agent and control capture agent can both bind the same control labeled agent, directly or indirectly. Such groupings of test set components generally can be determined in the context of particular embodiments of the disclosed method and apparatus and need not be based on permanent or absolute relationships between the control moderation capture agents, control capture agents, and control labeled agents.

I. Control Analyte (CA)

A control analyte (CA) can be any molecule, compound, composition or other agent that can compete either with a control moderation capture agent for binding to a control labeled agent (in the control MCA format) or with a control labeled agent for binding to a control moderation capture agent (in the control LA format). The control analyte generally is different from the analyte to prevent any cross reactivity between the analyte and control analyte as well as preventing any cross reactivity between the control analyte and the labeled agent, moderation capture agent, or test capture agent. In some embodiments, a control analyte can also be any molecule, compound, composition or other agent that can specifically affect the binding of the control labeled agent to the control moderation capture agent and/or the control capture agent and which is present in the test and used for the purpose of test validation.

J. Apparatuses

The disclosed apparatuses can be any apparatus that can be used to carry out all or part of any form of the disclosed methods. Useful apparatuses are devices designed or adapted to carry out all or part of any form of the disclosed methods. Useful devices include test cells. Test cells are devices that comprise a flow path through which liquid samples can flow. Generally the flow path of a test cell can comprise at least one moderation zone and at least one test zone. The test cell and flow path can be constructed of any suitable material. Generally the flow path can comprise a void space and/or a material through which liquid or fluid can flow. The test cell can consist of the flow path or can further comprise other elements and structures. For example, useful test cells comprise a body, which can be unitary or multipart, in which the flow path is disposed. The body can comprise one or more inlets where one or more fluids can be introduced to the test cell. Flow path material can also extend outside the body of the flow cell and such flow path material can be used to introduce fluid to the test cell.

Useful flow paths can comprise a substrate, such as a permeable material through which liquid or fluid can flow. For example, the flow path or substrate can comprise polymers, carbohydrates, fibers, filters, threads, particles, beads, gels, or any combination of these or other permeable materials. Many materials are known for mediating fluid flow and these materials can be used in and adapted for the disclosed test cells. The material for the flow path of a test cell can be selected based on the type of flow (for example, capillary flow, electrophoretic force, gravity (natural or artificial), and chromatographic flow). The flow path can be unitary or multipart. For example, flows paths can comprise a single substantially homogenous material, a single heterogeneous material, multiple different elements of the same materials, multiple different elements of different materials, or any combination. For example, a flow path can comprise a single element. Different components for use in the disclosed method can be disposed in different regions of this single element. As another example, a flow path can comprise a base material in which or on which one or more pads are located. In such embodiments, it is useful to provide different components for use in the disclosed method on different pads. For example, one or more pads can comprise or embody one or more moderation zones, one or more test zones, one or more control zones, one or more control moderation zones, one or more reservoir zones, and/or one or more sample zones.

Test cells can be made of any material on or in which substrate(s) can be disposed. For example, all types of polymers and plastic materials can be used to form test cells. Test cells can also be formed from material(s) that can form substrates for the flow path. In some forms of test cell, the test cell can consist of the flow path, with no other mounting material present.

An example of a useful test cell can have an elongate outer casing (the body of the test cell) which can house an interior permeable material capable of transporting an aqueous solution through the length of the test cell (the flow path of the test cell). The body can define a sample inlet, and interior regions which, for ease of description, can be designated as a test volume. The flow path can be disposed in the test volume. The interior region can also comprise an optional reservoir volume. The reservoir zone of the flow path can be disposed in the reservoir volume. If present, the reservoir volume can be disposed in a section of the test cell spaced apart from the inlet. The reservoir zone of the flow path can be, for example, a sorbent material. The reservoir zone can act to receive liquid transported along the flow path defined by the permeable material and extending from the inlet and through the test volume. The body of test cells can be closed save for one or more inlets for the introduction of fluids to the test cell. The body of test cells can also include one or more windows through which observations or measurements can be made of the flow path and components in the flow path. Such windows can be solid, open, transparent, opaque or in other condition. Detection need not require a window. For detection purposes all that is required is that measurement or detection of label be possible. Thus, for example, a visual observation can make use of a transparent or translucent window. A radiation or field detection by an instrument may not require any window if the radiation or field can penetrate the body of the test cell. Alternatively, the window can be made of a material transparent or translucent to the radiation or field to be detected. A window can also be an uncovered opening onto the flow path. A window can also be open or exposed or can be openably covered with some other material. Certain labels can be detected in situ due to the characteristics of the label, such as the electrical properties of semiconductor or metal colloids by measuring changes of conductivity or impedance within the strips at various zones.

The test volume and/or flow path can have a substantially constant cross-sectional shape and/or cross-sectional area. Alternatively, the test volume and/or flow path can include one or more changes in cross-sectional shape and/or cross-sectional area along the test volume and/or flow path. For example, the flow path can be restricted or narrowed in the moderation and test zones, thereby channeling and concentrating fluid flow into contact with the moderation and test zones. Test cells can also form or comprise one or more microfluidic channels or flow paths.

Test cells need not be enclosed or form a test volume. For example, test cells can comprise a surface on which one or more flow paths or substrates are disposed. A test strip is an example of such a test cell format. A test cell can also be formed of layers of material for any of the portions, including stacked and/or bundled layers of porous material comprising, for example, the same or various moderation capture agent within the moderation zone and/or stacked and/or bundled layers of porous material comprising test and/or control zones. Such layers and bundles can comprise both the body or support part of the test cell and/or the substrate or flow path of the test cell. Use of such layers can allow, for example, moderation zones or other zones to be constructed from several unbacked pads or strips (made of, for example, nitrocellulose membrane), whereby each moderation zone or other zone is a different pad and the pads are overlapped so that the sample flows through one moderation zone pad then the next one until the sample reaches and hydrates the test zone pad or strip. Such zone and test cell configurations allow, for example, manufacture of rolls of moderation zones from which pads or strips can be cut to form the multiple moderation zones of the test cell. This also allows easy manufacture of test cells having different amounts of various agents in the test cells. For example, by using one, two or three moderation zone pads in different test cells (where the pads all have the same moderation control agent), the test cells will contain one, two or three times as much of the moderation capture agent relative to each other. This is useful for optimizing or determining the proper amount of moderation capture agent to employ in a test set. The above arrangements of layers and pads can be used for any other zones in the test cell.

As with any test cell, the surface of a surface or strip form of test cell can be made of any material on which substrate(s) can be disposed. For example, all types of polymers and plastic materials can be used to form test cells. Test cells can also be formed from material(s) that can form substrates for the flow path. In some forms of test cell, the test cell can consist of the flow path, with no other mounting material present.

The test cell can also include one or more solution filtering materials (which can also be referred to as filters or filtration materials) disposed in the flow path. Such filters can be disposed anywhere in the flow path as may be useful or appropriate. For example, a filter can be disposed between an inlet and a moderation zone. Filters can be part of the flow path. Filters can comprise separate filter elements (such as a pad) disposed within the body of the test cell in fluid communication with the flow path, but can also be integrated into other materials of the flow path.

In some forms of the disclosed method, using a test cell, the method can be conducted by contacting a sample with the test cell. This can be done in a variety of ways including for example, placing the inlet of the test cell in contact with the sample. One then waits for the test sample to pass through the test cell, where the sample passes into reactive contact with the moderation zone(s) and test zone(s). In some embodiments, the labeled agent can be mixed with the sample and incubated briefly before being contacted with the test cell. In these embodiments, the liquid sample can be contacted with the test cell by placing the inlet of the test cell in contact with the sample or directly adding the liquid sample to the moderation zone of the test cell. In other embodiments, the labeled agent can be disposed in preserved form in the flow path within the test cell. The sample passes through the inlet and the interior of the test cell along the flow path past the moderation, test and control zones, where reactions take place as described elsewhere herein. The labeled agent-test capture agent conjugate that results in the presence of analyte comprises the immobilized test capture agent bound to the labeled agent. The presence of the labeled agent-test capture agent conjugate (and thus the presence of analyte in the sample) can be indicated by detection of label in the test zone.

By providing a reservoir of sorbent material disposed beyond the moderation, test, and control zones, a relatively large volume of liquid sample and any analyte it contains can be drawn through the flow path. Optionally, the region of the flow path in the test cell defining the moderation, test, and control zones can be restricted in cross-sectional area relative to other regions of the flow path. This feature produces a “bottle-neck” effect where all analyte in the entire volume of sample must pass through the restricted flow area immediately about the moderation and test zones.

Disclosed are devices for detecting one or more analytes. In the MCA format, the device can comprise one or more test cells, wherein each test cell comprises one or more substrates, wherein each substrate comprises one or more moderation zones and one or more test zones, wherein one or more moderation capture agents are immobilized in at least one of the moderation zones, wherein one or more test capture agents are immobilized in at least one of the test zones, wherein one or more liquid samples can flow through the substrate, wherein one or more labeled agents can be brought into contact with at least one of the samples, wherein at least one of the samples can flow into contact with at least one of the moderation capture agents, wherein the moderation capture agents can bind to one or more of the labeled agents, wherein each labeled agent comprises a label, wherein each labeled agent can bind to an analyte, wherein the labeled agent binds to the analyte if the analyte is present in the sample, wherein the moderation capture agents can bind to the labeled agent substantially only when the labeled agent is not bound to the analyte, wherein the sample can flow into contact with at least one of the test capture agents, wherein each test capture agent can bind to one or more of the labeled agents, wherein the labeled agents can accumulate at the moderation zone if the analyte is not present or is present in low amount, wherein labeled agents can accumulates at the test zone if the analyte is present, wherein accumulation of labeled agent at the site of the test capture agent indicates the presence of the analyte in the sample.

In the LA format, the device can comprise one or more test cells, wherein each test cell comprises one or more substrates, wherein each substrate comprises one or more moderation zones and one or more test zones, wherein one or more moderation capture agents are immobilized in at least one of the moderation zones, wherein one or more test capture agents are immobilized in at least one of the test zones, wherein one or more liquid samples can flow through the substrate, wherein one or more labeled agents can be brought into contact with at least one of the samples, wherein at least one of the samples can flow into contact with at least one of the moderation capture agents, wherein the moderation capture agent can bind to one or more of the labeled agents and to an analyte, wherein each labeled agent comprises a label, wherein the moderation capture agent binds to the analyte if the analyte is present in the sample, wherein the moderation capture agent can bind to the labeled agent substantially only when the moderation capture agent is not bound to the analyte, wherein the sample can flow into contact with at least one of the test capture agents, wherein each test capture agent can bind to one or more of the labeled agents, wherein the labeled agents can accumulate at the moderation zone if the analyte is not present or is present in low amount, wherein labeled agents can accumulates at the test zone if the analyte is present, wherein accumulation of labeled agent at the site of the test capture agent indicates the presence of the analyte in the sample.

The liquid sample can be brought into contact with the labeled agents by allowing or causing the sample to flow in the test cell into contact with the labeled agents. At least one of the labeled agents can be detachably localized or detachably immobilized to the substrate through which the sample flows, wherein contact by the sample with the labeled agents causes the labeled agent to detach and flow with the sample. At least one of the labeled agents can be in a dry state together with a mixture designed to release the labeled agent when rehydrated, wherein the mixture provides for stability of the labeled agent. The mixture can comprise sugar, such as sucrose, lactose, dextran, fructose, trehalose, other mono and disaccharides, or a combination; sugar derivative, such as sucralose or a sugar substitute, such as lactitol, maltitol, xylitol, aspartame, saccharine, tagalose, or a combination; starch or starch derivative, such as maltodextrin; protein or proteinacous material, such as bovine serum albumin (BSA), casein, hydrolyzed casein, peptides, or a combination; nucleic acid or oligonucleotide; detergent, such as a block co-polymer, Tetronic 904 (BASF), sodium sacrosine, Tween-20, or a combination; salt, such as sodium benzoate, sodium gluconate, potassium glutamate, or a combination; a polymer, such as polyvinyl alcohol, polyvinyl pyrrolidone, or a combination; buffer, such as Tris, Tricine, potassium phosphate, sodium citrate, sodium borate, potassium carbonate, MES, PIPES, HEPES, EPPS, or a combination; or any combination of these. Many other compounds and materials are know that can provide stability or flow and these can be used with the disclosed method. One or more of these materials can be in a liquid or amorphous state.

The liquid sample can be brought into contact with the labeled agents by mixing the sample with the labeled agents prior to allowing or causing the sample to flow in the test cell into contact with one or more moderation capture agents. At least one of the labeled agents can be incorporated into a pipette tip, a tube, or a sample collection liquid that is brought into contact with the sample. At least one of the labeled agents can be a freeze-dried pellet that is brought into contact with the sample. The sample can be brought into contact with a substrate through which the sample flows, wherein the sample can be mixed with the labeled agents prior to bringing the sample into contact with the substrate.

The moderation capture agents can be immobilized to the substrate through which the sample flows, wherein binding of labeled agent to moderation capture agent causes the labeled agent to be immobilized at the site of the moderation capture agent. The substrate can be a porous membrane, nitrocellulose, a pad, or one or more particles in the substrate that do not migrate through the test cell with the flow or which migrate at a rate slower than the rate of the flow. The test capture agents can be immobilized to the substrate through which the sample flows, wherein binding of labeled agent to test capture agent causes the labeled agent to be immobilized at the site of the test capture agent. The substrate can be a porous membrane, nitrocellulose, a pad, or one or more particles in the substrate that do not migrate through the test cell with the flow or which migrate at a rate slower than the rate of the flow. The test capture agent can comprise an antibody specific for the labeled agent, an antibody specific for the analyte, an antibody specific for any binding site on the labeled agent, an antibody that binds the labeled agent but not the analyte, an antibody that binds the analyte but not the labeled agent, an antibody that can bind any binding site of the analyte-labeled agent conjugate, an antibody with any combination of these binding specificities, or a combination. Accumulation of labeled agent at the site of the moderation capture agent can indicate the absence of the analyte in the sample.

The substrate can comprise one or more moderation zones and one or more test zones, wherein the moderation capture agents are immobilized in at least one of the moderation zones, wherein the test capture agents are immobilized in at least one of the test zones, wherein the moderation zone is upstream of the test zone relative to the flow of the sample. The substrate can further comprise one or more sample zones, wherein the sample is brought into contact with the substrate at the sample zone, wherein the sample zone is upstream of the moderation zone relative to the flow of the sample. The substrate can further comprise one or more label zones, wherein at least one of the labeled agents can be detachably localized or detachably immobilized to the substrate in at least one of the label zone, wherein contact by the sample with the labeled agents causes the labeled agents to detach and flow with the sample, wherein the label zone can be downstream of the sample zone, relative to the flow of the sample, wherein the label zone can be upstream of the moderation zone relative to the flow of the sample. The substrate can further comprise a reservoir, wherein the reservoir is downstream of the test zone relative to the flow of the sample. At least one absorbent material can be in capillary contact with the test zone to facilitate capillary flow from the moderation zone into the test portion. The reservoir can receive the sample that flows through the substrate. The substrate can comprise a permeable material. The moderation zone can be visible, observable, or detectable or not visible, observable, or detectable outside of the test cell.

In some embodiments, each moderation capture agent can comprise a binding region, wherein the binding region of each moderation capture agent can bind to one or more of the labeled agents. In some embodiments, each test capture agent can comprise a binding region, wherein the binding region of each test capture agent can bind to one or more of the labeled agents.

In some embodiments of the LA format, each labeled agent can further comprise a binding region, wherein the binding region of the labeled agent can bind to the analyte, wherein the binding region of the labeled agent binds to the analyte if the analyte is present in the sample. The moderation capture agent can comprise the analyte, a mimic of the analyte, a derivative of the analyte, or a combination. The labeled agent can comprise an antibody specific for the analyte.

In some embodiments of the MCA format, each moderation capture agent can comprise a binding region, wherein the binding region of each moderation capture agent can bind to one or more of the labeled agents and to an analyte. The labeled agent can comprise the analyte, a mimic of the analyte, a derivative of the analyte, or a combination.

The disclosed devices can also include controls and control formats. For example, the substrate can further comprise one or more control zones, wherein one or more control capture agents are immobilized in at least one of the control zones, wherein one or more control labeled agents can be brought into contact with at least one of the samples, wherein each control labeled agent comprises a label, wherein the sample can flow into contact with at least one of the control capture agents, wherein each control capture agent can bind to one or more of the control labeled agents.

Accumulation of control labeled agent at the site of the control capture agent can indicate the validity of the assay or test. In some embodiments, each control labeled agent can bind to a control analyte, wherein the control labeled agent binds to the control analyte if the control analyte is present.

The substrate can further comprise one or control more moderation zones, wherein one or more control moderation capture agents are immobilized in at least one of the moderation zones, wherein each control labeled agent can bind to a control analyte, wherein the control labeled agent binds to the control analyte if the control analyte is present, wherein the sample can flow into contact with at least one of the control moderation capture agents, wherein each control moderation capture agent can bind to one or more of the control labeled agents, wherein the control moderation capture agent can bind to the control labeled agent substantially only when the control labeled agent is not bound to the control analyte, wherein each control labeled agent can accumulate at the site of the control moderation capture agent if the control analyte is not present or is present in low amount, wherein each control labeled agent can accumulate at the site of the control capture agent if the control analyte is present.

The sample can be allowed or caused to flow into contact with one or more control moderation capture agents, wherein the control moderation capture agents are immobilized, wherein each control moderation capture agent can bind to one or more of the control labeled agents, wherein the control moderation capture agent can bind to the control labeled agent substantially only when the control labeled agent is not bound to the control analyte, wherein each control labeled agent accumulates at the site of the control moderation capture agent if the control analyte is not present or is present in low amount, wherein each control labeled agent accumulates at the site of the control capture agent if the control analyte is present.

The substrate can comprise porous material. The substrate can comprise more than one type of porous material. The sample zone can comprise glass fiber, a polyester material, cellulose or cellulose derivate, or a combination. The moderation zone and test zone can comprise nitrocellulose, nylon, or chemically treated material sufficient to bind the moderation capture agents and test capture agents or to inhibit the movement of the moderation capture agents and test capture agents bound to the moderation zone and test zone. At least one of the moderation capture agents can be bound to particles, wherein at least one of the test capture agents is bound to particles, wherein pores within the substrate permit little or no particle migration when the liquid sample flows through the substrate. The reservoir zone can comprise cellulose, sponge, desiccant, or other liquid sorbent material sufficient to absorb enough liquid to cause the sample to flow into the test zone.

The moderation zone and test zone can be treated with a material that blocks the binding of proteins or nucleic acids to the substrate in the moderation zone and test zone. The blocking material can comprise polymer, polyvinyl alcohol, protein, BSA, peptide, peptide mixture, hydrolyzed casein, detergent, Pluronic P103 (BASF), other blocking materials, or a combination. The moderation capture agents and test capture agents can be stabilized with sugar, sugar derivate, non-sugar sweetener, salt, detergent, or a combination thereof. The moderation capture agents and test capture agents can be stabilized with trehalose at a concentration of 0.1%-5%.

The disclosed devices can have a channel through which the sample flows. The sample can flow through such a channel by, for example, by capillary action or pumping. The channel can comprise a bottom channel and a side cavitation. The channel can also comprise a top channel. The channel can be hollow or can be filled, partially or fully, with, for example, a substrate. Moderation capture agents, test capture agents, control capture agents and control moderation capture agents can be immobilized on the wall of the channel. Labeled agents and control labeled agents can be detachably localized on the wall of the channel.

K. Antibodies

Antibodies can be used for many of the reagent components in the disclosed method and apparatus. For example, analytes, labeled agents, moderation capture agents, test capture agents, control labeled agents, control capture agents, control analytes and control moderation capture agents can all be antibodies and/or antigens that bind antibodies. As used herein, the term “antibody” encompasses, but is not limited to, whole immunoglobulin (i.e., an intact antibody) of any class.

As used herein, the term “antibody or fragments thereof” encompasses chimeric antibodies and hybrid antibodies, with dual or multiple antigen or epitope specificities, and fragments, such as F(ab′)2, Fab′, Fab and the like, including hybrid fragments. Thus, fragments of the antibodies that retain the ability to bind their specific antigens are contemplated. The term “monoclonal antibody” as used herein refers to an antibody obtained from a substantially homogeneous population of antibodies, i.e., the individual antibodies comprising the population are identical except for possible naturally occurring mutations that may be present in minor amounts. Monoclonal antibodies can be prepared using hybridoma methods or any other suitable method.

A variety of immunoassay formats, including the disclosed method, can be used to select antibodies that selectively bind with a particular analyte, labeled agent, binding agent of a labeled agent, moderation capture agent, test capture agent, control labeled agent, control capture agent, control analyte, and/or control moderation capture agent, including variants and fragments thereof. For example, solid-phase ELISA immunoassays are routinely used to select antibodies selectively immunoreactive with an analyte, labeled agent, binding agent of a labeled agent, moderation capture agent, test capture agent, control labeled agent, control capture agent, control analyte, and/or control moderation capture agent, including variants and fragments thereof. Antibodies from any source (such as from phage libraries or any other creation and/or selection methodology) can also be selected by using the disclosed method or test cell (or any other a lateral flow strip) to select those antibodies and reagents that function within the disclosed method and test cell. For example, such a method could employ a protein A or protein G labeled agent to select those antibodies that would bind to the moderating agent and which can be tested for its ability to compete for the analyte of interest using the MCA format.

L. Labels

Any molecule, compound, composition, moiety, other agent or portion thereof that produces, generates or mediates generation of a detectable signal can be used as a label in the disclosed components and method. Suitable labels include (but are not limited to) enzymes (such as alkaline phosphatase (AP) or horseradish peroxidase (HRP)), fluorescent labels, calorimetric labels, radioisotopes, chelating agents, dyes, colloidal gold, other colloidal metals, magnetic particles, semiconductor particles, chemical chain reaction initiators, ligands (such as biotin), up converting phosphor particles, chemiluminescent agents, mass labels, mass tags, molecular barcodes, quantum dots, nanoparticles, metal sols, and combinations of these (see Chapter 9, Harlow and Lane, Antibodies: A Laboratory Manual. 1988 and U.S. Pat. No. 4,313,734, the disclosure of which is incorporated herein by reference). Coded combinations of labels can be used as labels. Such label combinations, many of which are know, can be used to provide unique and distinguishable signals to different components. This can allow many different labeled agents, for example, to be used and distinguishably detected in the same assay or test cell.

Labels can be detected by any appropriate means, mode or manner. Generally, the mode or manner of detection can be based on the label used and the type of signal that the label generates. For example, some labels generate or produce radiation or a field that can be detected. Such radiation or field can be an added property of the label, can be a characteristic of the structure of the label, or both. Such radiation or field can also be generated by the combination of the label and some other component, or by a component that is associated with the label, generated by the label, or whose generation is mediated by the label. These latter modes can be referred to as indirect signal generation. Labels can also be detected by physical analysis of all or part of the label. For example, labels or portions of labels (or even entire labeled agents) can be analyzed by mass spectrometry. For this type of detection, useful labels can include mass tags, include multiple mass tags that can be distinguished form each other by mass spectrometry. Many types of signal, many methods for generating signals, and many methods of detecting signals are known and can be use with and adapted for the disclosed methods and apparatuses. Some signals can be detected visually and these and most other signals can be detected through the use of appropriate instruments. For example, the labels can be detected by visual observation of label development in appropriate zones in the test cell (for labels that produce a visual signal) and/or by the use of an instrument to detect, for example, any radiation or field that may be generated by the label. Certain labels, such as semiconductors, carbon colloid, and metal colloids, can be detected in situ by measuring changes in the electrical field as the label accumulates within the moderation capture or test capture zones. In another detection methodology, the accumulation of a label within the moderation capture zone or test capture zones can be detected or measured by changes in light wavelength, such as those employed in thin film assays (ThermoBiostar) or variations of an optical waveguide (Biocentrex).

Engineering principles involved in the synthesis of colored particle conjugates are also known. (See Horisberger, Evaluation of Colloidal Gold as a Cytochromic Marker for Transmission and scanning Electron Microscopy, Biol. Cellulaire, 36, 253-258 (1979); Leuvering et al, Sol Particle Immunoassay, J. Immunoassay 1 (1), 77-91 (1980), and Frens, Controlled Nucleation for the Regulation of the Particle Size in Monodisperse Gold Suspensions, Nature, Physical Science, 241, pp. 20-22 (1973), whose teachings are hereby incorporated by reference).

Examples of suitable fluorescent labels include fluorescein isothiocyanate (FITC), 5,6-carboxymethyl fluorescein, Texas red, nitrobenz-2-oxa-1,3-diazol-4-yl (NBD), coumarin, dansyl chloride, rhodamine, amino-methyl coumarin (AMCA), Eosin, Erythrosin, BODIPY®, Cascade Blue®, Oregon Green®, pyrene, lissamine, xanthenes, acridines, oxazines, phycoerythrin, macrocyclic chelates of lanthanide ions such as quantum dye™, fluorescent energy transfer dyes, such as thiazole orange-ethidium heterodimer, and the cyanine dyes Cy3, Cy3.5, Cy5, Cy5.5 and Cy7. Examples of other specific fluorescent labels include 3-Hydroxypyrene 5,8,10-Tri Sulfonic acid, 5-Hydroxy Tryptamine (5-HT), Acid Fuchsin, Alizarin Complexon, Alizarin Red, Allophycocyanin, Aminocoumarin, Anthroyl Stearate, Astrazon Brilliant Red 4G, Astrazon Orange R, Astrazon Red 6B, Astrazon Yellow 7 GLL, Atabrine, Auramine, Aurophosphine, Aurophosphine G, BAO 9 (Bisaminophenyloxadiazole), BCECF, Berberine Sulphate, Bisbenzamide, Blancophor FFG Solution, Blancophor SV, Bodipy F1, Brilliant Sulphoflavin FF, Calcien Blue, Calcium Green, Calcofluor RW Solution, Calcofluor White, Calcophor White ABT Solution, Calcophor White Standard Solution, Carbostyryl, Cascade Yellow, Catecholamine, Chinacrine, Coriphosphine O, Coumarin-Phalloidin, CY3.1 8, CY5.1 8, CY7, Dans (1-Dimethyl Amino Naphaline 5 Sulphonic Acid), Dansa (Diamino Naphtyl Sulphonic Acid), Dansyl NH—CH3, Diamino Phenyl Oxydiazole (DAO), Dimethylamino-5-Sulphonic acid, Dipyrrometheneboron Difluoride, Diphenyl Brilliant Flavine 7GFF, Dopamine, Erythrosin ITC, Euchrysin, FIF (Formaldehyde Induced Fluorescence), Flazo Orange, Fluo 3, Fluorescamine, Fura-2, Genacryl Brilliant Red B, Genacryl Brilliant Yellow 10GF, Genacryl Pink 3G, Genacryl Yellow 5GF, Gloxalic Acid, Granular Blue, Haematoporphyrin, Indo-1, Intrawhite Cf Liquid, Leucophor PAF, Leucophor SF, Leucophor WS, Lissamine Rhodamine B200 (RD200), Lucifer Yellow CH, Lucifer Yellow VS, Magdala Red, Marina Blue, Maxilon Brilliant Flavin 10 GFF, Maxilon Brilliant Flavin 8 GFF, MPS (Methyl Green Pyronine Stilbene), Mithramycin, NBD Amine, Nitrobenzoxadidole, Noradrenaline, Nuclear Fast Red, Nuclear Yellow, Nylosan Brilliant Flavin E8G, Oxadiazole, Pacific Blue, Pararosaniline (Feulgen), Phorwite AR Solution, Phorwite BKL, Phorwite Rev, Phorwite RPA, Phosphine 3R, Phthalocyanine, Phycoerythrin R, Poiyazaindacene Pontochrome Blue Black, Porphyrin, Primuline, Procion Yellow, Pyronine, Pyronine B, Pyrozal Brilliant Flavin 7GF, Quinacrine Mustard, Rhodamine 123, Rhodamine 5 GLD, Rhodamine 6G, Rhodamine B, Rhodamine B 200, Rhodamine B Extra, Rhodamine BB, Rhodamine BG, Rhodamine WT, Serotonin, Sevron Brilliant Red 2B, Sevron Brilliant Red 4G, Sevron Brilliant Red B, Sevron Orange, Sevron Yellow L, SITS (Primuline), SITS (Stilbene Isothiosulphonic acid), Stilbene, Snarf 1, sulpho Rhodamine B Can C, Sulpho Rhodamine G Extra, Tetracycline, Thiazine Red R, Thioflavin S, Thioflavin TCN, Thioflavin 5, Thiolyte, Thiozol Orange, Tinopol CBS, True Blue, Ultralite, Uranine B, Uvitex SFC, Xylene Orange, and XRITC.

Preferred fluorescent labels are fluorescein (5-carboxyfluorescein-N-hydroxysuccinimide ester), rhodamine (5,6-tetramethyl rhodamine), and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 run), Cy5 (652 nm: 672 nm), Cy5.5 (682 nm; 703 nm) and Cy7 (755 nm; 778 nm), thus allowing their simultaneous detection. Other examples of fluorescein dyes include 6-carboxyfluorescein (6-FAM), 2′,4′,1,4,-tetrachlorofluorescein (TET), 2′,4′,5′,7′,1,4-hexachlorofluorescein (HEX), 2′,7′-dimethoxy-4′,5′-dichloro-6-carboxyrhodamine (JOE), 2′-chloro-5′-fluoro-7′,8′-fused phenyl-1,4-dichloro-6-carboxyfluorescein (NED), and 2′-chloro-7′-phenyl-1,4-dichloro-6-carboxyfluorescein (VIC). Fluorescent labels can be obtained from a variety of commercial sources, including Amersham Pharmacia Biotech, Piscataway, N.J.; Molecular Probes, Eugene, Oreg.; and Research Organics, Cleveland, Ohio.

Additional labels of interest include those that provide for signal only when the probe with which they are associated is specifically bound to a target molecule, where such labels include: “molecular beacons” as described in Tyagi & Kramer, Nature Biotechnology (1996) 14:303 and EP 0 070 685 B1. Other labels of interest include those described in U.S. Pat. No. 5,563,037 and PCT Applications WO 97/17471 and WO 97/17076.

M. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits for detection of one or more analytes, the kit comprising one or more test cells comprising one or more labeled agents, one or more moderation capture agents, and one or more test capture agents. The kits also can contain one or more control labeled agents, one or more control capture agents, one or more control analytes and/or one or more control moderation capture agents.

N. Mixtures

Disclosed are mixtures formed by performing or preparing to perform the disclosed method. For example, disclosed are mixtures comprising one or more analytes, one or more labeled agents and one or more moderation captures agents; one or more labeled agents and one or more test capture agents; one or more analytes and one or more labeled agents; one or more labeled agents and one or more moderation capture agents; one or more test cells and one or more analytes; one or more test cells and one or more labeled agents; one or more test cells and one or more moderation capture agents; one or more test cells and one or more test capture agents; one or more test cells and one or more one or more analytes, one or more labeled agents and one or more moderation captures agents; one or more test cells and one or more one or more labeled agents and one or more test capture agents; one or more test cells and one or more one or more analytes and one or more labeled agents; and one or more test cells and one or more one or more labeled agents and one or more moderation capture agents.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

O. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated.

P. Data Structures and Computer Control

Disclosed are data structures used in, generated by, or generated from, the disclosed method. Data structures generally are any form of data, information, and/or objects collected, organized, stored, and/or embodied in a composition or medium. The results of one or more assays stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein.

### Method

Disclosed are methods for the detection of one or more analytes. The disclosed methods are particularly useful for detecting analytes in, for example, a liquid sample such as a biological or environmental sample. Virtually any analyte in any sample can be detected using the disclosed method. For example, proteins, peptides, antibodies, small molecules, metabolites, hormones, breakdown products, antigens, epitopes, nucleic acids, lipids, carbohydrates, sugars, cells, spores, viruses, bacteria, inorganic compounds, organic compounds, drugs, poisons, contaminants, and the like are analytes that can be detected with the disclosed methods and apparatuses. The disclosed method can be performed in numerous formats. For example, multiple analytes can be detected in the same, different, or combination of apparatuses; multiple analytes can be detected in the same, different or combination of assays; and multiple analytes can be detected simultaneously, sequentially, or in any temporal order. The disclosed method makes use of both a competitive assay element and a capture assay element. This combination has the benefit of producing a visible signal in the presence of an analyte while using the format of a competitive assay.

The disclosed method generally involves, in the presence of one or more samples suspected of containing one or more analytes, (1) binding of one or more labeled agents (LA) to one or more moderation capture agents (MCA) in the absence of one or more of the analytes and no binding or reduced binding of the labeled agents to the moderation capture agents in the presence of one or more of the analytes, and (2) binding of labeled agents that are not bound to moderation capture agents to one or more test capture agents (TCA). When reference is made to an analyte being absent for purposes of detection using the methods of the invention, it is intended that such reference covers not only situations in which analyte is not present in any absolute terms, but also where analyte is present in such a small amount as to be not detectable using the methods set forth herein. The labeled agent comprises a binding agent and a label. The label of the labeled agent that is bound to test capture agent can be detected. Such detection indicates the presence of one or more of the analytes in one or more of the samples because labeled agents become available to bind test capture agents when the labeled agents fail to bind moderation capture agents in the presence of the analytes. The first aspect of the method is the competitive assay element of the disclosed method and the second aspect is the capture assay element of the disclosed method.

In useful forms of the method, the moderation capture agents and test capture agents can be immobilized at different physical locations and they can be exposed to the combination of labeled agents and samples sequentially. For example, a liquid flow of combined sample and labeled agent can flow through one or more moderation zones (where one or more moderation capture agents can be immobilized) and then through one or more test zones (where one or more test capture agents can be immobilized). In a liquid flow format, the mobile components (for example, analytes and labeled agents) can be transported by any suitable means such as by capillary flow, electrophoretic force, gravity (natural or artificial), and chromatographic flow. The moderation capture agents can be immobilized in one or more moderation zones. The test capture agents can be immobilized in one or more test zones. In some forms of the method, the method can be performed using one or more test cells, where a liquid sample can flow along the cell and encounter the different components and immobilization areas in sequence. The samples can be brought into contact with test cells in an inlet, loading zone, or sample zone, which generally can be upstream of the moderation zones (which itself generally can be upstream of the test zones). An example of a useful test cell is a lateral flow test strip.

The disclosed method generally can be performed, and the disclosed apparatus generally can be configured, in two main formats. In one format, which can be referred to as the MCA/analyte competitive format (or MCA format), analyte and moderation capture agent (which can be similar to each other) compete with each other for binding to the labeled agent (for example, an antibody). Examples 1 and 3 illustrate this format. In the other format, which can be referred to as the LA/analyte competitive format (or LA format), analyte and labeled agents (which can be similar to each other) compete with each other for binding to the moderation capture agent (for example, an antibody). Examples 2 and 4 illustrate this format. In the LA format, an analyte (if present in a sample) competes with a labeled agent for binding to a moderation capture agent. Labeled agent binds to the moderation capture agent in the absence of analyte. In the presence of analyte, the analyte binds to the moderation capture agent, thus reducing or eliminating binding of the labeled agent to the moderation capture agent. Labeled agent that does not bind to moderation capture agent can then go on to bind test capture agent. Thus, detection of the labeled agent bound to test capture agent (that is, in the test zone) indicates the presence of analyte. An example of an embodiment of the LA format is shown in FIG. 3. Competition for binding can be accomplished by including molecules, elements or moieties in the labeled agents that are similar or identical to molecules, elements or moieties of the analyte. In the LA format, useful binding agents in the labeled agent can be the analyte, an analyte mimic or mimetic of the analyte, or a derivative of the analyte.

In the MCA format, an analyte (if present in a sample) competes with a moderation capture agent for binding to a labeled agent. Labeled agent binds to the moderation capture agent in the absence of analyte. In the presence of analyte, the analyte binds to the labeled agent, thus reducing or eliminating binding of the labeled agent to the moderation capture agent (the labeled agent cannot bind, or can only bind with less affinity, to the moderation capture agent when the labeled agent is bound to the analyte). Labeled agent that does not bind to moderation capture agent can then go on to bind test capture agent. Thus, detection of the labeled agent bound to test capture agent (that is, in the test zone) indicates the presence of analyte. An example of an embodiment of the MCA format is shown in FIG. 4. Competition for binding can be accomplished by including molecules, elements or moieties in the moderation capture agent that are similar or identical to molecules, elements or moieties of the analyte. In the MCA format, useful moderation capture agents can be the analyte, an analyte mimic or mimetic of the analyte, or a derivative of the analyte.

In the LA format, the method can comprise bringing into contact one or more liquid samples and one or more labeled agents, wherein each labeled agent comprises a label, wherein each labeled agent can bind to an analyte, wherein the labeled agent binds to the analyte if the analyte is present in the sample, allowing or causing the sample to flow into contact with one or more moderation capture agents, wherein the moderation capture agents are immobilized, wherein each moderation capture agent can bind to one or more of the labeled agents, wherein the moderation capture agent can bind to the labeled agent substantially only when the labeled agent is not bound to the analyte, allowing or causing the sample to flow into contact with one or more test capture agents, wherein the test capture agents are immobilized, wherein each test capture agent can bind to one or more of the labeled agents, wherein each labeled agent accumulates at the site of the moderation capture agent if the analyte is not present or is present in low amount, wherein each labeled agent accumulates at the site of the test capture agent if the analyte is present, wherein accumulation of labeled agent at the site of the test capture agent indicates the presence of the analyte in the sample.

In the MCA format, the method can comprise bringing into contact one or more liquid samples and one or more labeled agents, wherein each labeled agent comprises a label, allowing or causing the sample to flow into contact with one or more moderation capture agents, wherein the moderation capture agents are immobilized, wherein each moderation capture agent can bind to one or more of the labeled agents and to an analyte, wherein the moderation capture agent binds to the analyte if the analyte is present in the sample, wherein the moderation capture agent can bind to the labeled agent substantially only when the moderation capture agent is not bound to the analyte, allowing or causing the sample to flow into contact with one or more test capture agents, wherein the test capture agents are immobilized, wherein each test capture agent can bind to one or more of the labeled agents, wherein each labeled agent accumulates at the site of the moderation capture agent if the analyte is not present or is present in low amount, wherein each labeled agent accumulates at the site of the test capture agent if the analyte is present, wherein accumulation of labeled agent at the site of the test capture agent indicates the presence of the analyte in the sample.

The liquid sample can be brought into contact with the labeled agents by allowing or causing the sample to flow into contact with the labeled agents. At least one of the labeled agents can be detachably localized or detachably immobilized to a substrate through which the sample flows, wherein contact by the sample with the labeled agents causes the labeled agent to detach and flow with the sample. The labeled agents can be in a dry state together with a mixture designed to release the labeled agent when rehydrated or released when the sample is present, wherein the mixture provides for stability of the labeled agent. The mixture can comprise sugar, such as sucrose, lactose, dextran, fructose, trehalose, other mono and disaccharides, or a combination; sugar derivative, such as sucralose or a sugar substitute, such as lactitol, maltitol, xylitol, aspartame, saccharine, tagalose, or a combination; starch or starch derivative, such as maltodextrin; protein or proteinacious material, such as bovine serum albumin (BSA), casein, hydrolyzed casein, peptides, or a combination; nucleic acid or oligonucleotide; detergent, such as a block co-polymer, Tetronic 904 (BASF), sodium sacrosine, Tween-20, or a combination; salt, such as sodium benzoate, sodium gluconate, potassium glutamate, or a combination; a polymer, such as polyvinyl alcohol, polyvinyl pyrrolidone, or a combination; or any combination of these. Many other compounds and materials are know that can provide stability and these can be used with the disclosed method. At least one of the labeled agents can be not in a dry state. One or more of these materials can be in a liquid or amorphous state.

The liquid sample can be brought into contact with the labeled agents by mixing the sample with the labeled agents prior to allowing or causing the sample to flow into contact with one or more moderation capture agents. At least one of the labeled agents can be incorporated into a pipette tip, a tube, or a sample collection liquid that is brought into contact with the sample. At least one of the labeled agents can be a freeze-dried pellet that is brought into contact with the sample. The sample can be brought into contact with a substrate through which the sample flows, wherein the sample can be mixed with the labeled agents prior to bringing the sample into contact with the substrate.

The moderation capture agents can be immobilized to a substrate through which the sample flows, wherein binding of labeled agent to moderation capture agent causes the labeled agent to be immobilized at the site of the moderation capture agent. The substrate can be a porous membrane, nitrocellulose, a pad, or one or more particles in the substrate that do not migrate through the test cell with the flow or which migrate at a rate slower than the rate of the flow. The test capture agents can be immobilized to a substrate through which the sample flows, wherein binding of labeled agent to test capture agent causes the labeled agent to be immobilized at the site of the test capture agent. The substrate can be a porous membrane, nitrocellulose, a pad, or one or more particles in the substrate that do not migrate through the test cell with the flow or which migrate at a rate slower than the rate of the flow. The test capture agent can comprise an antibody specific for the labeled agent, an antibody specific for the analyte, an antibody specific for any binding site on the labeled agent, an antibody that binds the labeled agent but not the analyte, an antibody that binds the analyte but not the labeled agent, an antibody that can bind any binding site of the analyte-labeled agent conjugate, an antibody with any combination of these binding specificities, or a combination. Accumulation of labeled agent at the site of the moderation capture agent can indicate the absence of the analyte in the sample.

The sample can flow through a substrate, wherein a test cell comprises the substrate, wherein the substrate comprises one or more moderation zones and one or more test zones, wherein the moderation capture agents are immobilized in at least one of the moderation zones, wherein the test capture agents are immobilized in at least one of the test zones, wherein the moderation zone is upstream of the test zone relative to the flow of the sample. The substrate can further comprise one or more sample zones, wherein the sample is brought into contact with the substrate at the sample zone, wherein the sample zone is upstream of the moderation zone relative to the flow of the sample. The substrate can further comprise one or more label zones, wherein at least one of the labeled agents can be detachably localized or detachably immobilized to the substrate in at least one of the label zone, wherein contact by the sample with the labeled agents causes the labeled agents to detach and flow with the sample, wherein the label zone can be downstream of the sample zone, relative to the flow of the sample, wherein the label zone can be upstream of the moderation zone relative to the flow of the sample. The substrate can further comprise a reservoir, wherein the reservoir is downstream of the test zone relative to the flow of the sample. At least one absorbent material can be in capillary contact with the test zone to facilitate capillary flow from the moderation zone into the test portion. The reservoir can receive the sample that flows through the substrate. The substrate can comprise a permeable material. The moderation zone can be visible, observable, or detectable or not visible, observable, or detectable outside of the test cell.

The label can comprise colloidal gold, colloidal metal, colloidal carbon, colloidal selenium, one or more enzymes, magnetic particles, paramagnetic particles, one or more dyes, dyed particles, one or more semiconductor materials, fluorescent label, colored protein, chelating compound, chemical reaction initiator, radioactive molecule, up converting phosphor particle, latex particles, detectable compound, or combinations thereof. The labeled agent can comprise an antibody, protein, nucleic acid, peptide, polysaccharide, virus, bacteria, or cell. In some embodiments, each moderation capture agent can comprise a binding region, wherein the binding region of each moderation capture agent can bind to one or more of the labeled agents. In some embodiments, each test capture agent can comprise a binding region, wherein the binding region of each test capture agent can bind to one or more of the labeled agents.

In the MCA format, each labeled agent can further comprise a binding region, wherein the binding region of the labeled agent can bind to the analyte, wherein the binding region of the labeled agent binds to the analyte if the analyte is present in the sample. The moderation capture agent can comprise the analyte, a mimic of the analyte, a derivative of the analyte, or a combination. The labeled agent can comprise an antibody specific for the analyte.

In the LA format, each moderation capture agent can comprise a binding region, wherein the binding region of each moderation capture agent can bind to one or more of the labeled agents and to an analyte. The labeled agent can comprise the analyte, a mimic of the analyte, a derivative of the analyte, or a combination.

The disclosed method can also include controls and control formats. For example, the method can further comprise bringing into contact at least one of the liquid samples and one or more control labeled agents, wherein each control labeled agent comprises a label, allowing or causing the sample to flow into contact with one or more control capture agents, wherein the control capture agents are immobilized, wherein each control capture agent can bind to one or more of the control labeled agents.

Accumulation of control labeled agent at the site of the control capture agent can indicate the validity of the assay or test. In some embodiments, each control labeled agent can bind to a control analyte, wherein the control labeled agent binds to the control analyte if the control analyte is present.

The method can further comprise allowing or causing the sample to flow into contact with one or more control moderation capture agents, wherein the control moderation capture agents are immobilized, wherein each control moderation capture agent can bind to one or more of the control labeled agents, wherein the control moderation capture agent can bind to the control labeled agent substantially only when the control labeled agent is not bound to the control analyte, wherein each control labeled agent accumulates at the site of the control moderation capture agent if the control analyte is not present or is present in low amount, wherein each control labeled agent accumulates at the site of the control capture agent if the control analyte is present.

Accumulation of control labeled agent at the site of the control moderation capture agent in the absence of control analyte can indicate the validity of the assay or test. This can follow from the fact that interactions of the control analyte, control labeled agent, control moderation capture agent, and control capture agent can serve as analogs of the interactions of the analyte, labeled agent, moderation capture agent, and test capture agent in the test assay. For this reason, useful control components can be chosen to have interactions that are similar to the interactions of the main assay components. Accumulation of control labeled agent at the site of the control capture agent in the presence of control analyte can indicate the validity of the assay or test. Accumulation of control labeled agent at the site of the control moderation capture agents and at the site of the control capture agent in that presence of less control analyte than the amount of control labeled agent present can indicate the validity of the assay or test. These indications can follow from the fact that interactions of the control analyte, control labeled agent, control moderation capture agent, and control capture agent can serve as analogs of the interactions of the analyte, labeled agent, moderation capture agent, and test capture agent in the test assay. For this reason, useful control components can be chosen to have interactions that are similar to the interactions of the main assay components. In the case of a split in the binding of control labeled agent between the control moderation capture agent and control capture agent, the split can serve as a sensitive indicator that interactions are occurring as intended in the assay and are not being influenced by unusual conditions or adulteration of the sample. The sensitivity comes from the fact that a particular distribution of control labeled agent can be expected in the control moderation zone and in the control zone (this distribution can be validated beforehand in choosing the control components and their relative amounts). Any change in distribution indicates an alteration caused by conditions in the assay. For example, if a cross-linking agent is present in the sample, more of the control labeled agent will be bound (cross-linked) to the control moderation zone. If salt or pH conditions are unusual (such that interactions are weakened), less control labeled agent will bind to either zone.

The method can further comprise allowing or causing the sample to flow into contact with one or more control moderation capture agents, wherein the control moderation capture agents are immobilized, wherein each control moderation capture agent can bind to one or more of the control labeled agents and to a control analyte, wherein the control moderation capture agent binds to the control analyte if the control analyte is present, wherein the control moderation capture agent can bind to the control labeled agent substantially only when the control moderation capture agent is not bound to the control analyte, wherein each control labeled agent accumulates at the site of the control moderation capture agent if the control analyte is not present or is present in low amount, wherein each control labeled agent accumulates at the site of the control capture agent if the control analyte is present.

Accumulation of control labeled agent at the site of the control moderation capture agent in the absence of control analyte can indicate the validity of the assay or test. Accumulation of control labeled agent at the site of the control capture agent in the presence of control analyte can indicate the validity of the assay or test. Accumulation of control labeled agent at the site of the control moderation capture agents and at the site of the control capture agent in the presence of less control analyte than the amount of control labeled agent present can indicate the validity of the assay or test.

The method can further comprise allowing or causing the sample to flow into contact with one or more control moderation capture agents, wherein the control moderation capture agents are immobilized, wherein each control moderation capture agent can bind to one or more of the control labeled agents. There can be more labeled control agent present than control moderation capture agent, wherein accumulation of control labeled agent at the site of the control moderation capture agents and at the site of the control capture agent indicates the validity of the assay or test. The ratio of control labeled agent bound to control moderation capture agent and control labeled agent bound to control capture agent can be used to establish that the method is quantitative for the amount of labeled agent bound to test capture agent. The ratio of the amount of control labeled agent bound to control moderation capture agent to the amount of control labeled agent bound to control capture agent can be used to validate the assay or test.

The control analyte can be present in a known amount or in a standard amount, wherein the ratio of the amount of control labeled agent bound to control moderation capture agent to the amount of control labeled agent bound to control capture agent establishes a quantitative standard for the amount of labeled agent bound to the test capture agent. The control analyte can be present in the sample in a known amount or in a standard amount, wherein the ratio of the amount of control labeled agent bound to control moderation capture agent to the amount of control labeled agent bound to control capture agent establishes a quantitative standard for the amount of analyte in the sample.

The ratio of labeled agent bound to moderation capture agent and labeled agent bound to test capture agent can be used to quantitate the amount of analyte in the sample. An instrument can be used to detect label bound to the moderation capture agent and test capture agent.

The substrate can comprise porous material. The substrate can comprise more than one type of porous material. The sample zone can comprise glass fiber, a polyester material, a cellulose or cellulose derivate, or a combination. The moderation zone and test zone can comprise nitrocellulose, nylon, or chemically treated material sufficient to bind the moderation capture agents and test capture agents or to inhibit the movement of the moderation capture agents and test capture agents bound to the moderation zone and test zone. At least one of the moderation capture agents can be bound to particles, wherein at least one of the test capture agents is bound to particles, wherein pores within the substrate permit little or no particle migration when the liquid sample flows through the substrate. The reservoir zone can comprise cellulose, sponge, desiccant, or other liquid sorbent material sufficient to absorb enough liquid to cause the sample to flow into the test zone.

The moderation zone and test zone can be treated with a material that blocks the binding of proteins or nucleic acids to the substrate in the moderation zone and test zone. The blocking material can comprise polymer, polyvinyl alcohol, protein, BSA, peptide, peptide mixture, hydrolyzed casein, detergent, Pluronic P103 (BASF), other blocking materials, or a combination. The moderation capture agents and test capture agents can be stabilized with sugar, sugar derivate, non-sugar sweetener, salt, detergent, or a combination thereof. The moderation capture agents and test capture agents can be stabilized with trehalose at a concentration of 0.1%-5%.

### Specific Embodiments

Disclosed is a method to detect the presence or absence of at least one analyte in at least one sample comprising adding at least one liquid sample to at least one first portion of a test device, allowing the liquid sample to flow to a second portion of the permeable material termed a moderation zone, allowing the liquid sample to flow from the second portion of the permeable material to the third portion termed the test zone whereupon detection of at least one unbound conjugate can be detected. The test device can comprise at least one permeable material defining the at least one first portion, at least one second portion, and at least one third portion positioned so as to permit capillary flow communication between each first and second portion and between each second and third portion. The at least one of the first portion can be the site for application of at least one liquid sample, and for at least one conjugate movably supported therein, wherein the at least one conjugate can comprise at least one binder for at least one analyte coupled to at least one label comprising at least one labeled analyte binding compound. In the MCA format, the labeled analyte binding compound can be, for example, monoclonal anti-cocaine (or cocaine derivative) labeled with colloidal gold, monoclonal anti-human IgG labeled with colored latex particles, or monoclonal anti-hCG (alpha or beta subunit or alpha and beta subunit complex) labeled with a dye. At least one labeled conjugate (also termed labeled agent) can interact with at least one fixed ligand comprising the analyte, analyte mimic, or analyte derivative (also termed moderation capture agent). At least one conjugate can compete for binding with at least one fixed ligand together with any analyte(s) present in the liquid sample. For example, a moderation zone of cocaine or benzoylecgonine chemically coupled to BSA can be striped onto a nitrocellulose membrane that captures the colloidal gold labeled anti-cocaine monoclonal, unless the monoclonal binding is inhibited by the presence of cocaine in the sample (benzoylecgonine and cocaine can be used interchangeably in this example). Owing to at least partial inhibition of the conjugate binding to the moderation zone due to the presence of at least one analyte in the sample, and by subsequently reacting the mixture with at least one anti-conjugate binding compound fixed within the test zone region, at least one labeled conjugate can accumulate within the test zone in proportion to the concentration of at least one analyte in the sample being tested. This can be accomplished, for example, by using a test zone comprising of anti-mouse antibody from goat sprayed onto nitrocellulose, by using protein A and/or protein G sprayed onto nitrocellulose, or by using a monoclonal antibody that specifically recognizes the monoclonal anti-cocaine labeled with colloidal gold. One may also use an anti-BSA antibody if the labeled conjugate was blocked using BSA as described elsewhere in this method.

Also disclosed is a method to detect the presence or absence of at least one analyte in at least one sample comprising adding at least one liquid sample to at least one first portion of a test device comprising at least one permeable material defining at least one first portion, at least one second portion, and at least one third portion positioned so as to permit capillary flow communication between each first and second portion and between each second and third portion where the at least one first portion being the site for application of at least one liquid sample, and for at least one labeled analyte binding protein (labeled analyte binding protein is a form of labeled agent) movably supported therein, wherein the at least one labeled agent consists of at least one binder for at least one analyte coupled to at least one label comprising at least one labeled analyte binding compound (e.g. monoclonal anti-cocaine labeled with colloidal gold, monoclonal anti-human IgG labeled with colored latex particles, or monoclonal anti-hCG labeled with a dye; labeled analyte binding protein is a form of labeled agent), and allowing the liquid sample to flow to a second portion of the permeable material termed a moderation zone wherein at least one labeled agent interacts with at least one moderation capture agent (MCA) comprising the analyte, analyte mimic, or analyte derivative, wherein at least one labeled agent competes for binding with at least one fixed moderation capture agent together with any analyte(s) of interest present in the liquid sample (e.g. a moderation zone of cocaine or benzoylecgonine chemically coupled to BSA striped onto a nitrocellulose membrane that captures the colloidal gold labeled anti-cocaine monoclonal, unless the monoclonal binding is inhibited by the presence of cocaine in the sample), and allowing the liquid sample to flow from the second portion of the permeable material to the third portion termed the test zone whereupon detection of at least one unbound analyte-labeled agent conjugate can be detected, owing to at least partial inhibition of the labeled agent binding to the moderation zone due to the presence of at least one analyte in the sample, by subsequently reacting the mixture with at least one test capture agent (TCA) fixed within the test zone region whereby at least one analyte-labeled agent conjugate could accumulate within the test zone in proportion to the concentration of at least one analyte in the sample being tested (e.g. using a test zone comprising of anti-mouse antibody from goat sprayed onto nitrocellulose, using protein A and/or G sprayed onto nitrocellulose, using a monoclonal antibody that specifically recognizes the monoclonal anti-cocaine labeled with colloidal gold, or using an antibody that recognizes the blocking agent used to make the labeled agent).

Also disclosed is a device or kit to detect the presence or absence of at least one analyte in at least one sample comprising a test device comprising at least one permeable material defining at least one first portion, at least one second portion, and at least one third portion positioned so as to permit capillary flow communication between each first and second portion and between each second and third portion, the at least one first portion being the site for application of at least one liquid sample, and for at least one conjugate movably supported therein, wherein the at least one conjugate comprises at least one binder for at least one analyte coupled to at least one label comprising at least one labeled analyte binding compound. In use, the liquid sample can be allowed to flow to a second portion of the permeable material termed a moderation zone wherein at least one labeled conjugate interacts with at least one fixed ligand comprising of the analyte, analyte mimic, or analyte derivative wherein at least one conjugate competes for binding with at least one fixed ligand together with any analyte(s) present in the liquid sample. The liquid sample can then be allowed to flow from the second portion of the permeable material to the third portion termed the test zone whereupon at least one unbound conjugate can be detected. By at least partial inhibition of the conjugate binding to the moderation zone due to the presence of at least one analyte in the sample, and by subsequently reacting the mixture with at least one anti-conjugate binding compound fixed within the test zone region, at least one labeled conjugate can accumulate within the test zone in proportion to the concentration of at least one analyte in the sample being tested.

Also disclosed is a test device to detect the presence or absence of at least one analyte in at least one sample comprising adding at least one liquid sample to at least one first portion of a test device comprising at least one permeable material defining at least one first portion, at least one second portion, and at least one third portion positioned so as to permit capillary flow communication between each first and second portion and between each second and third portion where the at least one first portion being the site for application of at least one liquid sample, and for at least one labeled analyte binding protein (labeled agent) movably supported therein, wherein the at least one labeled agent consists of at least one binder for at least one analyte coupled to at least one label comprising at least one labeled analyte binding compound, and allowing the liquid sample to flow to a second portion of the permeable material termed a moderation zone wherein at least one labeled agent interacts with at least one moderation capture agent (MCA) comprising the analyte, analyte mimic, or analyte derivative, wherein at least one labeled agent competes for binding with at least one fixed moderation capture agent together with any analyte(s) of interest present in the liquid, and allowing the liquid sample to flow from the second portion of the permeable material to the third portion termed the test zone whereupon detection of at least one unbound analyte-labeled agent conjugate can be detected, owing to at least partial inhibition of the labeled agent binding to the moderation zone due to the presence of at least one analyte in the sample, by subsequently reacting the mixture with at least one test capture agent (TCA) fixed within the test zone region whereby at least one analyte-labeled agent conjugate could accumulate within the test zone in proportion to the concentration of at least one analyte in the sample being tested, whereby the ratio of the signal of the analyte-labeled agent conjugate within the moderation zone can be compared to the signal within the test zone used to capture the analyte-labeled agent conjugate as an indicator of the presence or absence of an analyte(s) of interest in the sample.

Also disclosed is a kit to detect the presence or absence of at least one analyte in at least one sample comprising adding at least one liquid sample to at least one first portion of a test device comprising at least one permeable material defining at least one first portion, at least one second portion, and at least one third portion positioned so as to permit capillary flow communication between each first and second portion and between each second and third portion where the at least one first portion being the site for application of at least one liquid sample, and for at least one labeled analyte binding protein (labeled agent) movably supported therein, wherein the at least one labeled agent consists of at least one binder for at least one analyte coupled to at least one label comprising at least one labeled analyte binding compound, and allowing the liquid sample to flow to a second portion of the permeable material termed a moderation zone wherein at least one labeled agent interacts with at least one moderation capture agent (MCA) comprising the analyte, analyte mimic, or analyte derivative, wherein at least one labeled agent competes for binding with at least one fixed moderation capture agent together with any analyte(s) of interest present in the liquid, and allowing the liquid sample to flow from the second portion of the permeable material to the third portion termed the test zone whereupon detection of at least one unbound analyte-labeled agent conjugate can be detected, owing to at least partial inhibition of the labeled agent binding to the moderation zone due to the presence of at least one analyte in the sample, by subsequently reacting the mixture with at least one test capture agent (TCA) fixed within the test zone region whereby at least one analyte-labeled agent conjugate could accumulate within the test zone in proportion to the concentration of at least one analyte in the sample being tested, whereby the ratio of the signal of the analyte-labeled agent conjugate within the moderation zone can be compared to the signal within the test zone used to capture the analyte-labeled agent conjugate as an indicator of the presence or absence of an analyte(s) of interest in the sample.

At least one absorbent material can be in capillary contact with the third zone to facilitate capillary flow from the second portion into the third portion. The conjugate can be in a dry or amorphous state together with a mixture designed to release the conjugate when rehydrated and which can provide for stability of the conjugate. The mixture can comprise at least one sugar such as sucrose, lactose, dextran, fructose, trehalose, or other mono and disaccharides. The mixture can comprise at least one sugar derivatives such as sucralose, or sugar substitutes such as lactitol, maltitol, xylitol, aspartame, saccharine, or tagalose. The mixture can comprise at least one starch or starch derivative such as maltodextrin. The mixture can comprise at least one protein or proteinacous materials such as bovine serum albumin (BSA), casein, hydrolyzed casein, peptides, and similar materials known by those skilled in the art. The mixture can comprise at least one nucleic acids or oligonucleotide. The mixture can comprise at least one detergent such as block co-polymers such as Tetronic 904 (BASF), sodium sacrosine, Tween-20, or similar detergents known by those skilled in the art. The mixture can comprise at least one salt such as sodium benzoate, sodium gluconate, or potassium glutamate. The mixture can comprise at least one polymer such as polyvinyl alcohol or polyvinyl pyrrolidone. The mixture can comprise at least one buffering agent such as Tris, Tricine, potassium phosphate, sodium citrate, sodium borate, potassium carbonate, MES, PIPES, HEPES, or EPPS.

The conjugate can be introduced into the sample being tested prior to introduction to the first portion, which can include incorporation of the conjugate into a pipette tip, a tube, a sample collection liquid, or as a freeze-dried pellet that can be mixed with the sample. For example, the conjugate, such as monoclonal anti-hCG colloidal gold, can be incorporated into the pipette tip or into the tube used to collect or to measure the volume of the urine sample thereby eliminating the need to use the label in the device. The labeled ligand binding compound can be dry or not. For example, a liquid capsule that can be dissolved when the sample can be introduced or can be mechanically broken to activate the labeled ligand binding compound.

At least one fixed ligand within the second zone can be bound to the porous membrane, including nitrocellulose, or affixed to particles that do not migrate or which migrate at a slower rate through the permeable material. For example, the moderation zone could be ligand affixed onto beads that can be too big to flow through the porous membrane thereby forming a moderation zone. At least one fixed anti-ligand binding compound within the third portion test zone can be bound to the porous membrane, including nitrocellulose, or affixed to particles that do not migrate or which migrate at a slower rate through the permeable material.

The second portion can be hidden from the field of view of the user. This can help keep users from getting confused if a line is seen before the test zone region. The second portion can be visible, for example, if an instrument is used to read the intensity of the test lines. For example, the ratio of the moderation zone line to the test zone line can allow quantitation and support test validity.

The conjugate label can comprise colloidal gold, colloidal metal, colloidal carbon, colloidal selenium, enzyme(s), magnetic or paramagnetic particles, dye(s), dyed particles, semiconductor material(s), fluorescent label, colored protein, chelating compound, radioactive molecule, up converting phosphor particle, latex particles, detectable compound, and/or chemical reaction initiator or combinations thereof. The conjugate can contain an antibody, protein, a nucleic acid, a peptide, polysaccharide, virus, bacteria, cell, or a combination thereof that provides binding to the analyte or to a component within the permeable membrane. The conjugate can be an antibody, the analyte itself or analyte derivate, a virus such as an antibody expressing phage, a bacteria such as staff A or staff G, a polysaccharide such as a lectin, a peptide, or a cell. The same material can be used in the moderation and test zones.

The test zone within the third portion can comprise an antibody against the conjugate, such as an anti-mouse antibody, and/or a species specific moiety such as protein A and/or protein G, and/or an anti-label material such as anti-FITC antibody, and/or a material that binds to a non-reaction substance used to make or which can be incorporated into the labeled ligand binding compound(s), such as an anti-BSA antibody when BSA is used to block colloidal gold particles used as a label. There can be multiple capture zones such as one that uses an antibody against the blocking agent used for a first drug, another nearby test capture zone against a different blocking agent used for a second drug, a generic capture reagent for monoclonals, another one for nucleic acid sequences for one or more of the tests, etc.

The second portion can comprise a ligand capable of binding at least one analyte of interest and the first portion contains at least one conjugate comprising of at least one analyte and/or analyte derivative. The analyte or analyte derivate can be on the labeled particle which competes for the antibody to the analyte sprayed onto the moderation zone of a nitrocellulose membrane. At least one labeled non-analyte control can be incorporated within the first portion and used to indicate the validity of the test method described by subsequent capturing of the reagent within the subsequent second and/or third portion of the test device. For example, a positive control such as ovalbumin labeled with colloidal gold that can be subsequently captured by a monoclonal anti-ovalbumin can be included.

A non-analyte labeled material can be introduced into the sample before introducing the sample into the first portion of the test device and subsequently captured in the second and/or third portion of the device. The control can be introduced when the sample is liquefied or added before use in the device. The label can be captured subsequently in the test validation zone or control zone. A moderation zone contained in the second portion can be used to partially capture some of the non-analyte labeled material with subsequent capture of some of the remaining non-analyte labeled material in the test validation zone contained within the third portion of the device. A nominal amount of material can be used in the moderation zone that will bind some of the control (50%, for example) and capture additional material in a later control or test validation zone. The ratio of the amount of both zones could be used as internal calibrators as well as test validation. An instrument can be used to read and quantify the signals. A visual read of sufficient label in the control zone would indicate the test worked. This method of test validation could reflect adulteration of the sample. For example, sometimes glutaraldehyde is used to crosslink the anti-drug antibodies to the drug conjugate on the nitrocellulose thereby indicating, in conventional assays, that there is no drug in the sample when in reality there may be. In the disclosed method, if the sample contains glutaraldehyde, it would crosslink the control material to the moderation test material thereby giving no line or signal in the validation test zone thereby indicating that the sample was adulterated. If on the other hand, one makes the sample extremely acid or basic or incorporates a strong detergent into the sample, the moderation zone would not capture any of the labeled material nor would the validation test zone thereby indicating the sample was again invalid.

The ratio of the amount of material captured in the test validation zone (control zone) can be used to establish a quantitative assay relative to the amount of labeled analyte binding compound captured in the test zone. The ratio of the amount non-analyte labeled material captured in the moderation zone to the amount of labeled material captured in the test validation zone (control zone) can be used to quantify or establish the test validity of the method.

More than one analyte can be detected, whereby one conjugate can be partially captured within the moderation zone contained in the second portion and partially captured in a subsequent third portion. The captured label can be used to help quantify another analyte tested at the same time or used to indicate test validity of the sample. For example, one could use the amount of amylase in a saliva sample, or even human serum albumin, as an internal control for the amount or concentration of the sample being tested. An internal control or calibrator can be used as one of the “analytes” measured. Thus the amount of signal for the human serum albumin (HSA) would be proportional to the dilution factor since HSA remains at a relatively constant level in blood and this could be used to compare against other signals such as the amount of drug signal measured thereby giving a more quantitative or semi-quantitative measurement of the level of a drug.

The ratio of the signal of the conjugate within the moderation zone can be compared to the signal within the test zone used to capture the conjugate as an indicator of the presence or absence of an analyte(s) in the sample. An instrument can be used to detect and measure the signal of the conjugate(s) in the moderation and/or test and/or control zones and thereby used to quantitate the amount of analyte present in the sample and/or provide semi-quantitative measurements and/or provide test validation data. For example, the moderation zone can be small (has less moderation capture agent) so that it captures only 50% of the labeled ligand binding compound without any analytes present and another zone further downstream (test zone) to capture the label then compare the signals generated from the moderation zone to the test zone. If an analyte is present, there will be decrease in the moderation zone and there should be a corresponding test zone increase in signal. This ratio can provide quantitation and test validation data.

The porous material can contains more than one type of porous material. The first potion can be comprised of glass fiber, a polyester material, cellulose or cellulose derivate, or other types of material practiced by those in the art. The second portion and third portion can comprise nitrocellulose, nylon, or chemically treated material sufficient to bind the fixed ligands or to inhibit the movement of the fixed ligands bound to particles that pores within the permeable membrane permit little or no particle migration when the liquid sample flows through the permeable membrane. The third portion, opposite the second portion, can comprise cellulose, sponge, desiccant, or other liquid sorbent material sufficient to absorb at enough of the liquid to cause sample to pass into the third portion.

The second and third portion can be treated with a material that blocks the binding of proteins or nucleic acids to the permeable material used within these the portions. The blocking material can comprise a polymer such as polyvinyl alcohol, a protein such as BSA, a peptide or peptide mixture such as hydrolyzed casein, a detergent such as Pluronic P103 (BASF), or other blocking materials or combination of materials known by those skilled in the art. The reagents of the second portion and third portion can be stabilized with a sugar, sugar derivate, non-sugar sweetener, salt, and/or detergent or combinations thereof in an amount to effectively provide a stabilizing effect on the reagents. Trehalose can be used at a concentration of 0.1%-5%.

### EXAMPLES

### A. Example 1

### Morphine Drug of Abuse Test

This is an example of the MCA/analyte competitive format of the disclosed method.

1. Preparation of Labeled Agent:

A 10 ml solution of 10 OD/ml colloidal gold (20-60 nanometer sized, such as Onasco's 3FA88.25-R or equivalent; this is the label) in a 50 mM pH 8.0 Tricine buffer (Sigma) can be prepared. While vortexing moderately, 50 μg of monoclonal anti-morphine antibody (Fitzgerald Industries P/N 10-M18 or equivalent; this is the binding agent) can be added to the mixture over a period of 10 seconds (equates to 0.5 ugm/OD/mL). The mixture can be stirred moderately for 5 minutes, then excess sites can be blocked by adding bovine serum albumin (Sigma) to a final concentration of 0.1% BSA and continue to stir for 10 minutes. Optimization can be empirically performed.

2. Preparation of the Control Labeled Agent:

In this example, a strepavidin colloidal gold conjugate can be used as a control labeled agent. A 10 ml solution of 10 OD/ml colloidal gold (20-60 nanometer size, such as Onasco's 3FA88.25-R or equivalent) can be prepared in a 100 mM MES pH 5.5 (Sigma) buffer. While vortexing moderately, 100 μg of streptavidin or streptavidin G′ (Sigma) can be added to the mixture over a period of 10 seconds (equates to 1 μg/OD/mL) generally at pH 4.5-5.5. The mixture can be stirred moderately for 5 minutes, then excess sites can be blocked by adding polyethylene glycol (2K-20K mw, Sigma) to a final of 0.05%-0.1% w/w and the mixture can be stirred for 10 minutes. Alternatively BSA or other material can be used to block, but these tend to be less stable at lower pH. This material must be titrated, just like the labeled agent above, but typical usage would be around 1 OD/mL and can be mixed with the labeled agent and labeled agent buffer described in the next step.

3. Preparation of Sample Pad (Portion One):

A 4× labeled agent dilution buffer (stabilizing and release mixture) can be prepared comprising 40% lactitol (Sigma or equivalent), 10% sodium saccharine, 4% block co-polymer detergent Tetronic 904 (BASF), 100 mM Tricine pH 8.0 (Sigma or equivalent), 0.4% polyvinyl alcohol (10 kd, Sigma or equivalent), 0.1% polyvinylpyrrolidone (Sigma or equivalent), and 1% potassium gluconate (Sigma or equivalent). Various dilutions of the labeled agent can be prepared from 4 OD/ml to 0.5 OD/ml into a 25% v/v dilution of the labeled agent dilution buffer making the difference up with water. Each mixture can be sprayed onto the edge of a sample pad (30 mm×280 mm glass fiber pad such as Lydal BIO-015) at 1.5 μl/mm using an Isoflow dispenser (Imagene Technologies) and dried. Each level represents a different cutoff level of the drug and the best level can be selected by empirical testing of the pads with the desired drug cutoff level. The labeled agent dilution buffer can also be adjusted more or less and the components adjusted more or less to achieve the desired cutoff. This can be done empirically. The correct amount of labeled agent and/or labeled agent diluent can be determined by assembling the materials and testing with samples containing morphine 50% above and 50% below the desired cutoff.

4. Preparation of the Test Cell:

The protective liner can be removed from the backing card used to hold the permeable membranes in place (80 mm×280 mm×0.015″ thick super white polystyrene with GL-87 adhesive from G&L Precision Die Cutting or equivalent) and a piece of Mylar-backed nitrocellulose 25×280 mm (Millipore SHF1350225 or equivalent) can be placed onto the adhesive backing with one edge of the membrane 28 mm from the side of the backing card with the Mylar side facing the adhesive layer and press into place. For one of the conjugate pads prepared above, the non-conjugate side can be abutted along the edge of the backing card that was 28 mm from the nitrocellulose and gently pressed into place whereby the colloidal gold conjugate edge overlapped the nitrocellulose by 2 mm thereby providing contact between the first portion and second portion of the test device that provides capillary movement of a sample from the sample pad (conjugate pad) onto the nitrocellulose second portion (moderation zone), to be applied in subsequent steps. An absorbent material (28 mm×280 mm EMI #30250) can then be placed along the top edge of the adhesive backed card overlapping the third portion of the nitrocellulose and abutting the top edge of the card and gently pressed into place thereby providing a reservoir for the liquid sample to flow into from the third zone.

5. Preparation of Moderation Zone:

A moderation zone comprising morphine-BSA (Fitzgerald Industries P/N 80-IM50 or equivalent) at 2 mg/mL in 2% trehalose (Sigma) and 10 mM TABS pH 9.0 (Sigma) can be applied at 0.1 ul/mm at a position about 2-4 mm from the edge of the conjugate release pad, yet out of the viewing area of the test and control lines when the strips are cut and assembled into a device, using an Imagene Isoflow dispenser setup according to manufacturer's instructions. If using a multiple pump version of the dispenser, this material can be stripped at the same time as the test line and control line reagents are applied (below). Note: In some cases, one can stripe more than one moderation line or include multiple moderation capture agents into one or more lines, including a moderation capture agent for the control line (which is referred to herein as a control moderation capture agent), should one desire to provide a standard by which to titrate against. In that case, one can stripe a second (control) moderation line comprising a control moderation capture agent following similar steps to those described above. In the case of multiple moderation zones, a wider nitrocellulose membrane (i.e. 35 mm wide) can be used along with narrower sample and/or absorbent sink pads that still allow capillary flow from one type of membrane into the next one.

6. Preparation of Test Zone:

In this example, the test zone can comprise a goat anti-mouse antibody (Sigma or equivalent) diluted at 2 mg/mL into 2% trehalose (stabilizer) and 10 mM TABS buffer (pH 9.0, Sigma Chemical Company) and striped at 0.05 μl/mm at the desired location of the test zone (between 3-6 mm from the moderation zone) using an Imagene Isoflow striper. This can be done simultaneously with application of the moderation and/or control zones. More than one test zone can also be used for each different analyte. For example, one could use a monoclonal that specifically recognizes only the anti-morphine monoclonal and another that only recognizes a monoclonal anti-cocaine. Alternatively, one can employ differing blocking agents in making the conjugate particles whereby one is blocked with BSA and another blocked with ovalbumin then capture each unbound conjugate using a monoclonal anti-BSA or monoclonal anti-ovalbumin in different test zone lines. This can also be expanded to include using oligonucleotides as test capture agents and complementary oligonucleotides coupled to different conjugates so as to detect different analytes at the same time within the same sample. As described above, the more analytes being tested may require wider lengths of nitrocellulose and correspondingly shorter widths of sample and sink pads.

7. Preparation of the Control Zone:

In this example, a strepavidin colloidal gold can be employed as a control labeled agent and the control zone employed a biotinylated protein, BSA (Pierce Chemical 1858747) formulated at 1 mg/mL in 2% trehalose and 10 mM TABS (pH 9.0, Sigma) and striped in the control zone region, 3-6 mm above the last test zone region, at 0.05 μl/mm using an Imagine Isoflow dispenser or equivalent. This control region can be applied at the same time as the moderation and/or test zone reagents if using a multiple pump dispenser.

8. Creation of Test Cells:

Once the reagents are applied, the cards can be dried overnight in a room <40% relative humidity and at 37-45° C. then cut into strips with a slitter (Kinematic 2360 or equivalent) and placed into plastic test housings (Aptech or equivalent).

9. Performing the Assay:

Using 4 mm wide test cells, a sample volume of 75-150 μl can be applied to the sample pad (glass fiber in this case) where it rehydrates the labeled agent and control labeled agent then flows by capillary action onto the nitrocellulose material where it interacts with the moderation zone. In selecting the appropriate concentration of labeled agent to use, one can select those pads that contain the highest concentration of morphine conjugate that completely binds to the moderation zone, depending upon the desired morphine cutoff desired. If morphine is present in the sample, normally at about 300 ng/mL, it competes for the morphine conjugate thereby reducing the conjugate binding to the moderation zone and subsequently being captured in the test zone, which captures all unbound morphine conjugate that proceeds past the moderation zone, thereby giving an increasing signal (label) as it accumulates in the test zone region until enough label is present to detect visually or using an instrument to read the label. The control labeled agent simply flows along the test strip and is eventually captured in the control zone where the streptavidin binds to the biotin coupled of the labeled CA, in this case BSA, indicating that the test components worked.

### B. Example 2

### Reverse-Orientation Morphine Drug of Abuse Test

This is an example of the LA/analyte competitive format of the disclosed method.

1. Preparation of Labeled Agent:

In this example, morphine-BSA (Fitzgerald Industries P/N 80-IM50 or equivalent) can be coupled to a colloidal gold carrier at 0.5 μg/OD/mL under the same conditions as described in Example 1, except that the colloidal gold conjugate is centrifuged at 10,000×g for 5 minutes, washed with 10× volume of 25% conjugate dilution buffer, then resuspended in the 25% conjugate dilution buffer to remove any unbound BSA used to block active protein binding sites on the colloidal gold. Again, various concentrations can be added to the same conjugate release/stabilizing buffer along with the streptavidin control conjugate and used in the assay to empirically determine the best amounts of conjugate and best conditions under which it performs at the desired cutoff.

2. Preparation of the Control Labeled Agent:

Preparation of the control labeled agent can be performed as in Example 1.

3. Preparation of Sample Pad:

Preparation of the sample pad can be performed as in Example 1.

4. Preparation of Test Cell:

Preparation of the test cell can be performed as in Example 1.

5. Preparation of Moderation Zone:

In this example, the moderation capture agent used in the moderation zone can be a monoclonal antibody against morphine (Fitzgerald Industries P/N 10-M18 or equivalent) following the same striping procedures described in example 1.

6. Preparation of Test Zone:

In this example, the test capture agent can be a monoclonal or polyclonal anti-BSA that used to capture the BSA backbone of the morphine-BSA colloidal gold test conjugate that is does not bind to the moderation zone due to the presence of morphine in the sample.

7. Preparation of Control Zone:

Preparation of the control zone can be performed as in Example 1.

8. Preparation of Test Cells:

Preparation of the control zone can be performed as in Example 1.

9. Performing the Assay:

The assay can be performed as in Example 1.

### C. Example 3

### Detection of Antibodies to HIV-1

This is an example of the MCA/analyte competitive format of the disclosed method.

1. Preparation of Labeled Agent:

In this example, the analyte being detected are antibodies to HIV-1 in a clinical sample. To detect the analyte this test utilizes the conserved HIV-1 gp41 immunodominant (IDR)* peptide chemically coupled to a BSA carrier molecule using EDAC or other cross linking agents used by those skilled in the art and commercially available from Pierce Chemical Company as a labeled agent. A 10 mL solution of 10 OD/mL colloidal gold (20-60 nanometer sized, such as Onasco's 3FA88.25-R or equivalent) can be prepared in a 50 mM pH 8.0 Tricine buffer (Sigma). The sample can then be vortexed moderately, and 100 μg of the gp41-IDR-BSA can be added to the mixture over a period of 10 seconds (equates to 1 ugm/OD/mL). The sample can continue to be stirred moderately for 5 minutes, then excess sites can be blocked by adding polyethylene glycol (2K-20K mw, Sigma) to a final of 0.05%-0.1% w/w and continued to be stirred for 10 minutes. Every preparation can be different. Each can be optimized for the pH under which it binds to the label, in this case colloidal gold, and each can be optimized as to the amount of ligand per OD colloidal gold used. Optimization can be empirically done.

2. Preparation of the Control Labeled Agent

Preparation of the control labeled agent can be performed as in Example 1.

3. Preparation of Sample Pad:

A 4× conjugate dilution buffer (stabilizing and release mixture) can be prepared comprising 40% lactitol (Sigma or equivalent), 10% sodium saccharine, 4% block co-polymer detergent Tetronic 904 (BASF), 100 mM Tricine pH 8.0 (Sigma or equivalent), 0.4% polyvinyl alcohol (10 kd, Sigma or equivalent), 0.1% polyvinylpyrrolidone (Sigma or equivalent), and 1% potassium gluconate (Sigma or equivalent; Note: this salt concentration can be adjusted or the salt changed for different stringencies depending upon the antibody clones used). Various dilutions of the conjugate can be made from 4 OD/mL to 0.5 OD/mL into a 25% v/v dilution of the conjugate dilution buffer making the difference up with water. Each mixture can be sprayed onto the edge of a sample pad (30 mm×280 mm glass fiber pad such as Lydal BIO-015) at 1.5 μl/mm using an Isoflow dispenser (Imagene Technologies) and dried. Each level represents a different conjugate level and the best level can be selected by empirical testing of the pads within the device using seroconverter samples. The conjugate dilution buffer can be adjusted more or less and the components adjusted more or less to achieve the desired cutoff. This can be done empirically. The correct amount of conjugate and/or conjugate diluent can be determined by assembling the materials and testing with negative and positive samples.

4. Preparation of Test Cell:

Preparation of the test cell can be performed as in Example 1.

5. Preparation of Moderation Zone:

In this example, the moderation zone can be a custom monoclonal made against the gp41-IDR peptide. A moderation zone can be striped comprising monoclonal anti-gp41-IDR (custom made) at 2 mg/mL in 2% trehalose (Sigma) and 10 mM TABS pH 9.0 (Sigma) can be applied at 0.1 ul/mm at a position about 2-4 mm from the edge of the conjugate release pad, yet out of the viewing area of the test and control lines when the strips are cut and assembled into a device, using an Imagene Isoflow dispenser setup according to manufacturer's instructions. If using a multiple pump version of the dispenser, this material can be striped at the same time as the test line and control line reagents are applied (below). In some cases, one can stripe more than one moderation line or include multiple moderation capture agents into one or more lines, including a moderation capture agent for the control line, should one desire to provide a standard by which to titrate against. In that case, one can stripe a second moderation line comprising an anti-gp41-IDR following similar steps to those described above. In the case of multiple moderation zones, a wider nitrocellulose membrane (i.e. 35 mm wide) can be used along with narrower sample and/or absorbent sink pads that still allow capillary flow from one type of membrane into the next one.

6. Preparation of Test Zone:

In this example, the test capture agent will need to capture the HIV antibody inhibited gp41-IDR conjugate. Since antibodies must bind to the conjugate to prevent its binding to the monoclonal anti-gp41, this example can employ the use of recombinant protein A to capture all antibodies in the sample, including those that have the labeled conjugate attached to them. Thus, the test zone can comprise of a recombinant protein A (Repligen) diluted at 2 mg/mL into 2% trehalose (stabilizer) and 10 mM TABS buffer (pH 9.0, Sigma Chemical Company) and can be striped at 0.05 μl/mm at the desired location of the test zone (usually 3-6 mm from the moderation zone) using an Imagene Isoflow striper. This can be done simultaneously with application of the moderation and/or control zones. More than one test zone can also be used for each different analyte. Alternatively, a test capture agent can be employed to capture the peptide backbone, the blocking agent, or a superficial agent employed within the conjugate and used simply as a marker or capture point for unbound conjugate.

7. Preparation of Control Zone:

Preparation of the control zone can be performed as in Example 1.

8. Creation of Test Cells:

Preparation of the control zone can be performed as in Example 1.

9. Performing the Assay:

For a 4 mm wide test strip, a typical sample volume of 75-150 μl can be applied to the sample pad (glass fiber in this case) where it rehydrates the control and test conjugates then flows by capillary action onto the nitrocellulose material where it interacts with the moderation zone. If the sample does not contain HIV-1 antibodies that recognize the gp41 peptide, then the gp41-IDR conjugate will bind to the moderation zone where it is captured by monoclonal anti-gp41-IDR. In selecting the appropriate concentration of labeled agent to use, one can typically select those pads that contain the highest concentration of labeled agent that completely binds to the moderation zone. If antibodies are present in the sample they compete for the gp41-IDR conjugate thereby reducing the conjugate binding to the moderation zone and subsequently being captured in the test zone, which captures all unbound analyte-labeled agent conjugate that proceeds past the moderation zone, thereby giving an increasing signal (label) as it accumulates in the test zone until enough label is present to detect visually or using an instrument to read the label. The control labeled agent simply flows along the test strip and is eventually captured in the control zone where the streptavidin binds to the biotin coupled to the binding agent of the control labeled agent, in this case BSA, indicating that the test components worked.

### D. Example 4

### Reverse-Orientation Detection of HIV-1 Antibodies

This is an example of the LA/analyte competitive format of the disclosed method.

1. Preparation of Labeled Agent:

In this example, the labeled agent comprises of a monoclonal anti-gp41-IDR* coupled to colloidal gold following the same protocol as described in Example 3 using the same concentrations, etc.

2. Preparation of the Control Labeled Agent:

Preparation of the control labeled agent can be performed as in Example 1.

3. Preparation of Sample Pad:

Preparation of the Sample Pad can be performed as in Example 3.

4. Preparation of Test Cell:

Preparation of the test cell can be performed as in Example 1.

5. Preparation of Moderation Zone:

In this example, the moderation zone is the HIV-1 gp41-IDR* chemically coupled to BSA (custom) that competes for the monoclonal anti-gp41 conjugate (label) with any antibodies against HIV-1 present in the sample that bind to the immunodominant region of gp41. A moderation zone can be striped comprising gp41-IDR-BSA (custom made) at 2 mg/mL in 2% trehalose (Sigma) and 10 mM TABS pH 9.0 (Sigma) applied at 0.1 ul/mm at a position about 2-4 mm from the edge of the conjugate release pad, yet out of the viewing area of the test and control lines when the strips are cut and assembled into a device, using an Imagene Isoflow dispenser setup according to manufacturer's instructions. If using a multiple pump version of the dispenser, this material can be striped at the same time as the test line and control line reagents are applied (below). In some cases, one can stripe more than one moderation line or include multiple moderation capture agents into one or more lines, including a moderation capture agent for the control line, should one desire to provide a standard by which to titrate against. In that case, one can stripe a second moderation line comprising gp41-IDR-BSA following similar steps to those described above. In the case of multiple moderation zones, a wider nitrocellulose membrane (i.e. 35 mm wide) can be used along with narrower sample and/or absorbent sink pads that still allow capillary flow from one type of membrane into the next one.

6. Preparation of Test Zone:

In this example, the test capture agent will need to capture unbound monoclonal anti-gp41-IDR conjugate that was inhibited from binding to the moderation zone because the sample contained antibodies against gp41 that competed for the gp41-BSA within the moderation zone. Since the conjugate is comprised on monoclonal antibodies, this example can employ the use of recombinant protein A to capture all antibodies, including unbound monoclonal anti-gp41 IDR conjugate. Thus, the test zone can comprise a recombinant protein A (Repligen) diluted at 2 mg/mL into 2% trehalose (stabilizer) and 10 mM TABS buffer (pH 9.0, Sigma Chemical Company) and striped at 0.05 μl/mm at the desired location of the test zone (usually 3-6 mm from the moderation zone) using an Imagene Isoflow striper. This can be done simultaneously with application of the moderation and/or control zones. More than one test zone can also be used for each different analyte, providing that a specific test capture agent is employed for each conjugate. Alternatively, a test capture agent can be employed to capture the peptide backbone, the blocking agent, or a superficial agent employed within the conjugate and used simply as a marker or capture point for unbound conjugate.

7. Preparation of Control Zone:

Preparation of the control zone can be performed as in Example 1.

8. Creation of Test Cells:

Preparation of the control zone can be performed as in Example 1.

9. Performing the Assay:

For a 4 mm wide test strip, a typical sample volume of 75-150 μl can be applied to the sample pad (glass fiber in this case) where it rehydrates the control and test conjugates then flows by capillary action onto the nitrocellulose material where it interacts with the moderation zone. If the sample does not contain HIV-1 antibodies that recognize the gp41 peptide, then the monoclonal anti-gp41-IDR conjugate will bind to the moderation zone where it is captured by gp41 IDR-BSA. In selecting the appropriate concentration of conjugate to use, one can select those pads that contain the highest concentration of conjugate that completely binds to the moderation zone. If antibodies are present in the sample they compete for the gp41-IDR-BSAe within the moderation zone thereby reducing the monoclonal anti-gp41-IDR conjugate binding to the moderation zone and subsequently being captured in the test zone, which captures all unbound labeled agent that proceeds past the moderation zone, thereby giving an increasing signal (label) as it accumulates in the test zone region until enough label is present to detect visually or using an instrument to read the label. The control labeled agent simply flows along the test strip and is eventually captured in the control zone where the streptavidin binds to the biotin coupled to the binding agent of the control labeled agent, in this case BSA, indicating that the test components worked.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a labeled agent” includes a plurality of such labeled agents, reference to “the labeled agent” is a reference to one or more labeled agents and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps.

Those skilled in the art will recognize, or be able to ascertain using no more than routine experimentation, many equivalents to the specific embodiments of the method and compositions described herein. Such equivalents are intended to be encompassed by the following claims.

I claim:

1. A method for detecting one or more analytes, the method comprising
bringing into contact one or more liquid samples and one or more labeled agents, wherein each labeled agent comprises a label, wherein each labeled agent can bind to an analyte, directly or indirectly, wherein the labeled agent binds to the analyte if the analyte is present in the sample,
allowing or causing the sample to flow into contact with one or more moderation capture agents, wherein the moderation capture agents are immobilized, wherein each moderation capture agent can bind to one or more of the labeled agents directly or indirectly, wherein the moderation capture agent can bind to the labeled agent when the labeled agent is not bound to the analyte,
allowing or causing the sample to flow into contact with one or more test capture agents, wherein the test capture agents are immobilized, wherein each test capture agent can bind to one or more of the labeled agents directly or indirectly,
wherein each labeled agent accumulates at the site of the moderation capture agent if the analyte is not present or is present in a non-detectable amount, wherein each labeled agent accumulates at the site of the test capture agent if the analyte is present, wherein accumulation of labeled agent at the site of the test capture agent indicates the presence of the analyte in the sample.

2. A method for detecting one or more analytes, the method comprising
bringing into contact one or more liquid samples and one or more labeled agents, wherein each labeled agent comprises a label,
allowing or causing the sample to flow into contact with one or more moderation capture agents, wherein the moderation capture agents are immobilized, wherein each moderation capture agent can bind to one or more of the labeled agents and to an analyte directly or indirectly, wherein the moderation capture agent binds to the analyte if the analyte is present in the sample, wherein the moderation capture agent can bind to the labeled agent when the moderation capture agent is not bound to the analyte,
allowing or causing the sample to flow into contact with one or more test capture agents, wherein the test capture agents are immobilized, wherein each test capture agent can bind to one or more of the labeled agents directly or indirectly,
wherein each labeled agent accumulates at the site of the moderation capture agent if the analyte is not present or is present in a non-detectable amount, wherein each labeled agent accumulates at the site of the test capture agent if the analyte is present, wherein accumulation of labeled agent at the site of the test capture agent indicates the presence of the analyte in the sample.

3. The method of claims 1 or 2, wherein at least one of the labeled agents is detachably localized on a substrate through which the sample flows, wherein contact by the sample with the labeled agents causes the labeled agent to detach and flow with the sample.

4. The method of one of claims 1 or 2, wherein at least one of the labeled agents is in a dry state together with a mixture designed to release the labeled agent when rehydrated, wherein the mixture provides for stability of the labeled agent.

5. The method of one of claims 1 or 2, wherein at least one of the labeled agents is not in a dry state.

6. The method of one of claims 1 or 2, wherein the liquid sample is brought into contact with the labeled agents by mixing the sample with the labeled agents prior to allowing or causing the sample to flow into contact with one or more moderation capture agents.

7. The method of one of claims 1 or 2, wherein the moderation capture agents are immobilized to a substrate through which the sample flows, wherein binding of labeled agent to moderation capture agent causes the labeled agent to be immobilized at the site of the moderation capture agent.

8. The method of one of claims 1 or 2, wherein the test capture agents are immobilized to a substrate through which the sample flows, wherein binding of labeled agent to test capture agent causes the labeled agent to be immobilized at the site of the test capture agent.

9. The method of one of claims 1 or 2, wherein the test capture agent comprises an antibody specific for the labeled agent, an antibody specific for the analyte, or both.

10. The method of one of claims 1 or 2, wherein accumulation of labeled agent at the site of the moderation capture agent indicates the absence of the analyte in the sample.

11. The method of one of claims 1 or 2, wherein the test cell comprises one or more moderation zones and one or more test zones, wherein the moderation capture agents are immobilized in at least one of the moderation zones, wherein the test capture agents are immobilized in at least one of the test zones, wherein the moderation zone is upstream of the test zone relative to the flow of the sample.

12. The method of claim 11, wherein the test cell further comprises one or more sample zones, wherein the sample is brought into contact with the test cell at the sample zone, wherein the sample zone is upstream of the moderation zone relative to the flow of the sample.

13. The method of claim 12, wherein the test cell further comprises one or more label zones, wherein at least one of the labeled agents is detachably localized on the test cell in at least one of the label zone, wherein contact by the sample with the labeled agents causes the labeled agents to detach and flow with the sample, wherein the label zone is downstream of the sample zone, relative to the flow of the sample, wherein the label zone is upstream of the moderation zone relative to the flow of the sample.

14. The method of one of claims 1 or 2, wherein the label comprises colloidal gold, colloidal metal, colloidal carbon, colloidal selenium, one or more enzymes, magnetic particles, paramagnetic particles, one or more dyes, dyed particles, one or more semiconductor materials, fluorescent label, colored protein, chelating compound, chemical reaction initiator, radioactive molecule, up converting phosphor particles, detectable compounds, or combinations thereof.

15. The method of one of claims 1 or 2 further comprising
bringing into contact at least one of the liquid samples and one or more control labeled agents, wherein each control labeled agent comprises a label,
allowing or causing the sample to flow into contact with one or more control capture agents, wherein the control capture agents are immobilized, wherein each control capture agent can bind to one or more of the control labeled agents.

16. The method of one of claims 1 or 2, wherein the ratio of labeled agent bound to moderation capture agent and labeled agent bound to test capture agent is used to quantitate the amount of analyte in the sample.

17. The method of one of claims 1 or 2, wherein an instrument is used to detect label bound to the moderation capture agent and test capture agent.

18. A device for detecting one or more analytes in a liquid sample, wherein the device is made up of one or more test cells with each test cell comprising at least one flow path through which the liquid sample can flow, said flow path comprising:
A) at least one inlet port for introducing one or more analyte-containing liquid samples into the flow path,
B) a means for bringing the liquid samples into contact with one or more labeled agents wherein each labeled agent comprises a labeled portion and a binding portion having the ability to bind an analyte in the liquid sample, directly or indirectly,
C) one or more moderation zones comprising one or more immobilized moderation capture agents containing one or more binding portions which can bind one or more of the labeled agents, directly or indirectly, when the labeled agent is not bound to an analyte in the liquid sample, such that labeled agent accumulates in the moderating zone if analyte is not present in the liquid sample in a detectable amount,
D) one or more test zones comprising one or more immobilized test capture agents wherein each test capture agent has a binding portion that can bind, directly or indirectly, to one or more labeled agents such that labeled agent accumulates in the test zone when analyte is present in the liquid sample in detectable amount,
E) a means for detecting the accumulation of labeled agent in the test zone, whereby the accumulation of labeled agent indicates the presence of analyte in the liquid sample.

19. A device for detecting one or more analytes in a liquid sample, wherein the device is made up of one or more test cells with each test cell comprising at least one flow path through which the liquid sample can flow, said flow path comprising:
A) at least one inlet port for introducing one or more analyte-containing liquid sample into the flow path,
B) a means for bringing the liquid sample into contact with one or more labeled agents, wherein each labeled agent comprises a labeled portion and a binding portion having the ability to bind to the moderation zone, directly or indirectly,
C) one or more moderation zones comprising one or more immobilized moderation capture agents containing one or more binding portions which can bind one or more of the labeled agents and to analyte, directly or indirectly, in the liquid sample, such that labeled agent accumulates in the moderating zone if analyte is not present in the liquid sample in a detectable amount,
D) one or more test zones comprising one or more immobilized test capture agents wherein each test capture agent has a binding portion that can bind, directly or indirectly, to one or more labeled agents such that labeled agent accumulates in the test zone then analyte is present in the liquid sample in detectable amount,
E) a means for detecting the accumulation of labeled agent in the test zone, whereby the accumulation of labeled agent indicates the presence of analyte in the liquid sample.

20. A device for detecting one or more analytes, the device comprising
One or more test cells, wherein each test cell comprises one or more moderation zones and one or more test zones, wherein one or more moderation capture agents are immobilized in at least one of the moderation zones, wherein one or more test capture agents are immobilized in at least one of the test zones,
wherein one or more liquid samples can flow through the test cell, wherein one or more labeled agents can be brought into contact with at least one of the samples,
wherein at least one of the samples can flow into contact with at least one of the moderation capture agents, wherein the moderation capture agents can bind to one or more of the labeled agents directly or indirectly, wherein each labeled agent comprises a label, wherein each labeled agent can bind to an analyte directly or indirectly, wherein the labeled agent binds to the analyte if the analyte is present in the sample, wherein the moderation capture agents can bind to the labeled agent when the labeled agent is not bound to the analyte,
wherein the sample can flow into contact with at least one of the test capture agents, wherein each test capture agent can bind to one or more of the labeled agents directly or indirectly,
wherein the labeled agents can accumulate at the moderation zone if the analyte is not present or is present in a non-detectable amount, wherein labeled agents can accumulates at the test zone if the analyte is present, wherein accumulation of labeled agent at the site of the test capture agent indicates the presence of the analyte in the sample.

21. A device for detecting one or more analytes, the device comprising
one or more test cells, wherein each test cell comprises one or more moderation zones and one or more test zones, wherein one or more moderation capture agents are immobilized in at least one of the moderation zones, wherein one or more test capture agents are immobilized in at least one of the test zones,
wherein one or more liquid samples can flow through the test cell, wherein one or more labeled agents can be brought into contact with at least one of the samples,
wherein at least one of the samples can flow into contact with at least one of the moderation capture agents, wherein the moderation capture agent can bind to one or more of the labeled agents and to an analyte directly or indirectly, wherein each labeled agent comprises a label, wherein the moderation capture agent binds to the analyte if the analyte is present in the sample, wherein the moderation capture agent can bind to the labeled agent when the moderation capture agent is not bound to the analyte,
wherein the sample can flow into contact with at least one of the test capture agents, wherein each test capture agent can bind to one or more of the labeled agents directly or indirectly,
wherein the labeled agents can accumulate at the moderation zone if the analyte is not present or is present in a non-detectable amount, wherein labeled agents can accumulates at the test zone if the analyte is present, wherein accumulation of labeled agent at the site of the test capture agent indicates the presence of the analyte in the sample.

22. The device of one of the claims 18, 19, 20 or 21, wherein the moderation zone is upstream of the test zone relative to the flow of the sample.

23. The device of one of claims 18, 19, 20 or 21, wherein the liquid sample is brought into contact with the labeled agents by allowing or causing the sample to flow into contact with the labeled agents.

24. The device of claim 23, wherein the test cell further comprises a substrate, wherein the sample flows through the substrate, wherein at least one of the labeled agents is detachably localized on the substrate through which the sample flows, wherein contact by the sample with the labeled agents causes the labeled agent to detach and flow with the sample.

25. The device of one of claims 18, 19, 20 or 21, wherein the labeled agent comprises an antibody, protein, nucleic acid, peptide, polysaccharide, virus, bacteria, or cell.

26. The device of one of claims 18, 19, 20 or 21, wherein each moderation capture agent comprises a binding region, wherein the binding region of each moderation capture agents can bind to one or more of the labeled agents.

27. The device of one of claims 18, 19, 20 or 21, wherein each test capture agent comprises a binding region, wherein the binding region of each test capture agent can bind to one or more of the labeled agents.

28. The device of claim 20, wherein each labeled agent further comprises a binding region, wherein the binding region of the labeled agent can bind to the analyte, wherein the binding region of the labeled agent binds to the analyte if the analyte is present in the sample.

29. The device of claim 20, wherein the moderation capture agent comprises the analyte, a mimic of the analyte, a derivative of the analyte, or a combination.

30. The device of claim 20, wherein the labeled agent comprises an antibody specific for the analyte.

31. The device of claim 21, wherein each moderation capture agent comprises a binding region, wherein the binding region of each moderation capture agent can bind to one or more of the labeled agents and to an analyte.

32. The device of claim 21, wherein the labeled agent comprises the analyte, a mimic of the analyte, a derivative of the analyte, or a combination.

33. The device of one of claims 18, 19, 20 or 21, wherein the test cell further comprises one or more control zones, wherein one or more control capture agents are immobilized in at least one of the control zones,
wherein one or more control labeled agents can be brought into contact with at least one of the samples, wherein each control labeled agent comprises a label,
wherein the sample can flow into contact with at least one of the control capture agents, wherein each control capture agent can bind to one or more of the control labeled agents.

34. The device of one of claims 18, 19, 20 or 21, wherein the ratio of labeled agent bound to moderation capture agent and labeled agent bound to test capture agent is used to quantitate the amount of analyte in the sample.

35. The device of one of claims 18, 19, 20 or 21, wherein an instrument is used to detect label bound to the moderation capture agent and test capture agent.

36. A kit for detection of one or more analytes, the kit comprising one or more test cells, wherein each test cell comprises one or more moderation zones and one or more test zones, wherein one or more moderation capture agents are immobilized in at least one of the moderation zones, wherein one or more test capture agents are immobilized in at least one of the test zones.

37. The kit of claim 36 further comprising one or more labeled agents.

38. The kit of claim 36, wherein the test cell further comprises one or more sample zones, wherein the samples can be brought into contact with the test cell at the sample zones, wherein the sample zone is upstream of the moderation zone relative to the flow of a sample through the test cell.

39. The kit of claim 36, wherein the test cell further comprises a reservoir, wherein the reservoir is downstream of the test zone relative to the flow of a sample through the test cell.

40. The kit of claim 36, wherein the test cell further comprises a substrate, wherein the sample flows through the substrate, wherein the substrate comprises a permeable material.

41. The kit of claim 36, wherein the test cell further comprises a substrate, wherein the sample flows through the substrate, wherein the moderation zone is not visible outside of the test cell.

42. The method of one of claims 1 or 2, wherein the sample flows through a channel.

43. The method of claim 42, wherein the sample flows by capillary action.

44. The device of one of claims 18, 19, 20 or 21, wherein the sample flows through a channel.

45. The device of claim 44, wherein the sample flows by capillary action.

46. The device of one of claims 18, 19, 20 or 21, wherein the substrate comprises beads or particles, wherein the beads or particles do not flow or flow at a slower rate that the sample.

47. The device of one of claims 18, 19, 20 or 21, wherein the labeled agent further comprises a marker, wherein the test capture comprises an antibody specific for the marker.

48. The method of one of claims 1 or 2, wherein the labeled agent further comprises a marker, wherein the test capture comprises an antibody specific for the marker.
